(19) United States
(12) Patent Application Publication
(10) Pub. No.: US 2025/0025445 A1
(43) Pub. Date: Jan. 23, 2025

(54) METHOD OF CARDIOPROTECTION

(71) Applicant: Race Oncology LTD., Sydney, New South Wales (AU)

(72) Inventors: Daniel TILLETT, Woollahra (AU); Doan Thi Minh NGO, Eleebana (AU); Aaron Leonid SVERDLOV, Eleebana (AU)

(21) Appl. No.: 18/711,415

(22) PCT Filed: Nov. 18, 2022
(86) PCT No.: PCT/AU2022/051380

§ 371 Date: May 17, 2024

(30) Foreign Application Priority Data
Nov. 19, 2021 (AU) 2021903734
Jun. 30, 2022 (AU) 2022901827

(51) Int. Cl. A61K 31/4168; A61K 31/4965; A61K 31/704; A61K 38/07; A61P 9/00

(52) U.S. Cl. CPC A61K 31/4168; A61K 31/4965; A61K 31/704; A61K 38/07; A61P 9/00

(57) ABSTRACT

The present invention relates to methods comprising administering to the subject an effective amount of a cardioprotective agent comprising bisantrene or a derivative thereof, or a pharmaceutically acceptable salt of bisantrene or derivative thereof.

### TECHNICAL FIELD

The present disclosure relates to a method for preventing or reducing cardiotoxicity in a subject, and to pharmaceutical compositions and kits for preventing or reducing cardiotoxicity in a subject.

### BACKGROUND

Cardiotoxicity is damage to cells of the cardiac muscle caused by cardiotoxic agents such as cardiotoxic chemotherapeutic agents. Cardiotoxicity can lead to conditions such as blood pressure changes, thrombosis, electrocardiographic changes, arrhythmias, myocarditis, pericarditis, myocardial infarction, cardiomyopathy, cardiac failure (left ventricular failure) and congestive heart failure.

A number of chemotherapeutic drugs for the treatment of cancer and other conditions also exhibit significant cardiotoxicity.

Drugs which are most associated with cardiotoxicity include anthracyclines, some tyrosine kinase inhibitors (TKIs) and some biologics based on monoclonal antibodies.

Anthracyclines are widely used chemotherapeutic agents in the treatment of cancer. The anthracyclines are among the most effective class of anticancer drugs developed so far and are effective against more types of cancer than many other classes of chemotherapeutic agent. Cancers treated with anthracyclines include leukemias, lymphomas, breast, stomach, uterine, ovarian, bladder, and lung cancers. Anthracycline drugs include, for example, doxorubicin, daunorubicin, epirubicin, valrubicin, mitoxantrone and idarubicin.

Despite their effectiveness, the use of anthracyclines as chemotherapeutic agents is limited by their cardiotoxicity. The anthracyclines exhibit a dose dependent and cumulative cardiotoxicity, and as a consequence, a maximum recommended cumulative dose must be set to prevent the development of adverse cardiac events, such as, for example, congestive heart failure. For example, McGowan et al. (2017) Cardiovascular Drugs and Therapy, 31 (1): 63-75 reports that the incidence of congestive heart failure is 4.78, 26% and 48% respectively when patients received doxorubicin at 400 mg/m², 550 mg/m² and 700 mg/m². Therefore, the lifetime cumulative doxorubicin exposure is limited to 400-450 mg/m² in order to reduce congestive heart failure incidence to less than 58, although variation in terms of tolerance to doxorubicin exists between individuals (Ewer & Ewer (2015), Nature Reviews Cardiology. 12 (9): 547-558). Although slightly less cardiotoxic, a similar situation exists for daunorubicin. As a consequence of the requirement to limit the cumulative exposure to anthracyclines, the full potential of these drugs for treating cancer may not be achieved. In addition, even at current restricted dosages, cardiac damage can nonetheless be incurred in some patients.

In addition to anthracyclines, some tyrosine kinase inhibitors (TKIs) and antibody-based therapeutics have also been reported to be cardiotoxic. TKIs such as imatinib mesylate (Gleevec), Nilotinib (Tasigna), sorafenib (Nexavar), sunitinib (Sutent) and dasatinib (Sprycel) have been reported to cause cardiotoxicity (Chu et al., Lancet (2007) 370:2011-2019; Xu et al., Hematol Rev. (2009) Marl; 1 (1): e4; Kerketla et al., Nature Medicine (2006) 12:908-916). The antibody based-therapeutic Trastuzumab is an example of a monoclonal antibody that can cause cardiotoxicity.

Proteasome inhibitors are also known to have cardiotoxic side-effects. For example, carfilzomib is a tetrapeptide epoxyketone and an analog of epoxomicin used as an anti-cancer medication, for multiple myeloma, acting as a selective irreversible proteasome inhibitor. However, carfilzomib can result in cardiotoxicity in up to 10% of patients and due to this cardiotoxicity is not used in solid tumours like breast cancer. There have also been multiple reports suggesting cardiotoxicity of bortezomib (Velcade®).

Risk factors may predispose a patient to cardiotoxicity. These include, for example: cumulative dose of a cardiotoxic agent; course of administration of a cardiotoxic agent; rate of administration of a cardiotoxic agent; concurrent administration of cardiotoxic agents; prior treatment with a cardiotoxic agent; and history of or pre-existing cardiovascular disorders.

A number of cardioprotective strategies have been explored, including the use of, for example, modified anthracyclines, statins, cardioselective beta-blockers and angiotensin antagonists. However, results have been mixed in humans.

It would be advantageous to develop improved cardioprotective strategies which reduce or prevent cardiotoxicity caused by cardiotoxic agents.

### SUMMARY OF THE INVENTION

Surprisingly, the present studies have shown that bisantrene, an anthracycline-like anthracene previously known to have antitumorigenic properties, is not only toxic to cancer cells, either alone, or in an at least additive, if not synergistic way when combined with other chemotherapeutic agents, but also mitigates the toxicity of cardiotoxic agents, including anthracyclines, the antineoplastic monoclonal antibody trastuzumab, and the proteasome inhibitors carfilzomib and bortezomib, to human cardiomyocytes, even though it is mildly toxic to human cardiomyocytes itself.

These properties of bisantrene allow for: increased efficacy of treatment of subjects using cardiotoxic therapeutic drugs with reduced cardiotoxicity; reduced dosing of cardiotoxic therapeutic drugs whilst achieving substantially the same therapeutic efficacy as achieved with full dosing (but no bisantrene). These benefits are quite important especially with use of drugs like anthracyclines which have lifetime dosing limits. The enhanced antineoplastic activity and avoidance of cardiotoxicity provided by bisantrene should also allow broader use of chemotherapeutic drugs like carfilzomib which has historically been too toxic to use in cancers outside of multiple myeloma.

Thus, a first aspect of the present invention provides a method for preventing or reducing drug-induced cardiotoxicity in a subject caused by a cardiotoxic agent, the method comprising administering to the subject an effective amount of a cardioprotective agent comprising bisantrene or a derivative thereof, or a pharmaceutically acceptable salt of bisantrene or derivative thereof.

An alternative first aspect provides a cardioprotective agent comprising bisantrene or a derivative thereof, or a pharmaceutically acceptable salt of bisantrene or derivative thereof, for use in preventing or reducing drug-induced cardiotoxicity in a subject caused by a cardiotoxic agent; or use of a cardioprotective agent comprising bisantrene or a derivative thereof, or a pharmaceutically acceptable salt of bisantrene or derivative thereof, in the manufacture of a medicament for preventing or reducing drug-induced cardiotoxicity in a subject caused by a cardiotoxic agent.

A second aspect provides a method for preventing or reducing cardiotoxicity induced or exacerbated by a cardiotoxic agent in a subject, the method comprising administering to said subject an effective amount of a cardioprotective agent comprising bisantrene or a derivative thereof, or a pharmaceutically acceptable salt of bisantrene or derivative thereof, prior to, simultaneously with, or after administration of the cardiotoxic agent.

An alternative second aspect provides a cardioprotective agent comprising bisantrene or a derivative thereof, or a pharmaceutically acceptable salt of bisantrene or derivative thereof, for use in preventing or reducing cardiotoxicity induced or exacerbated by a cardiotoxic agent in a subject; or use of a cardioprotective agent comprising bisantrene or a derivative thereof, or a pharmaceutically acceptable salt of bisantrene or derivative thereof, in the manufacture of a medicament for preventing or reducing cardiotoxicity induced or exacerbated by a cardiotoxic agent in a subject.

A third aspect provides a method for treating cancer in a subject, the method comprising administering:
a. a cardiotoxic chemotherapeutic agent in an amount that is therapeutically effective against the cancer; and
b. a cardioprotective agent comprising bisantrene or a derivative thereof, or a pharmaceutically acceptable salt of bisantrene or derivative thereof, in an amount that is cardioprotective.

An alternative third aspect provides a cardioprotective agent comprising bisantrene or a derivative thereof, or a pharmaceutically acceptable salt of bisantrene or derivative thereof, and a cardiotoxic chemotherapeutic agent, for use in treating cancer in a subject; or use of a cardioprotective agent comprising bisantrene or a derivative thereof, or a pharmaceutically acceptable salt of bisantrene or derivative thereof, and a cardiotoxic chemotherapeutic agent, in the manufacture of a medicament for treating cancer in a subject.

A fourth aspect provides a method of preventing or reducing cardiotoxicity in a subject being treated with a cardiotoxic chemotherapeutic agent, the method comprising administering to the subject an effective amount of a cardioprotective agent comprising bisantrene or a derivative thereof, or a pharmaceutically acceptable salt of bisantrene or derivative thereof.

An alternative fourth aspect provides a cardioprotective agent comprising bisantrene or a derivative thereof, or a pharmaceutically acceptable salt of bisantrene or derivative thereof, for use in preventing or reducing cardiotoxicity in a subject being treated with a cardiotoxic chemotherapeutic agent; or use of a cardioprotective agent comprising bisantrene or a derivative thereof, or a pharmaceutically acceptable salt of bisantrene or derivative thereof, in the manufacture of a medicament for preventing or reducing cardiotoxicity in a subject being treated with a cardiotoxic chemotherapeutic agent.

A fifth aspect provides a pharmaceutical composition comprising a cardioprotective agent comprising bisantrene or a derivative thereof, or a pharmaceutically acceptable salt of bisantrene or derivative thereof, and a cardiotoxic therapeutic agent, the composition having reduced cardiotoxicity compared to a composition comprising the cardiotoxic therapeutic agent but not comprising said cardioprotective agent.

A sixth aspect provides a pharmaceutical composition comprising: (a) a cardioprotective agent comprising bisantrene or a derivative thereof, or a pharmaceutically acceptable salt of bisantrene or derivative thereof; and (b) a cardiotoxic chemotherapeutic agent.

A seventh aspect provides a kit for preventing or reducing drug-induced cardiotoxicity in a subject, comprising: (a) a cardioprotective agent comprising bisantrene or a derivative thereof, or a pharmaceutically acceptable salt of bisantrene or derivative thereof; and (b) a cardiotoxic therapeutic agent.

An eighth aspect provides a kit for treating cancer, comprising: (a) a cardioprotective agent comprising bisantrene or a derivative thereof, or a pharmaceutically acceptable salt of bisantrene or derivative thereof; and (b) a cardiotoxic chemotherapeutic agent.

### BRIEF DESCRIPTION OF THE DRAWINGS

FIG. 1 shows primary human cardiomyocyte cell viability when cultured in the presence of 0-5 μM bisantrene alone, or when cultured also in the presence of 1 μM doxorubicin or 1M Carfilzomib for: (A) 24 hours; (B) 48 hours; and (C) 72 hours.

FIG. 2 shows primary human cardiomyocyte cell viability when cultured in the presence of: 1 μM doxorubicin alone; 1 μM doxorubicin and 500 nM trastuzumab; 1 μM doxorubicin, 500 nM trastuzumab and 1 μM bisantrene; 1 μM doxorubicin and 1 μM bisantrene; 1 μM doxorubicin, 500 nM trastuzumab and 5 μM bisantrene; and 1 μM doxorubicin, 500 nM trastuzumab and 5 μM bisantrene.

FIG. 3 shows MCF-7 breast cancer cell viability when cultured in the presence of 0-10 μM bisantrene alone, or when cultured also in the presence of 1 μM doxorubicin or 1 μM carfilzomib for 72 hours.

FIG. 4 shows H929 multiple myeloma cell viability when cultured in the presence of 0-5 μM bisantrene alone, or when cultured also in the presence of 1 μM carfilzomib for 72 hours.

FIG. 5 shows primary human cardiomyocyte cell viability when cultured in the presence of 1 μM bisantrene, in the presence of 1 μM daunorubicin, or in the presence of 1 μM bisantrene and 1 μM daunorubicin for 72 hours.

FIG. 6 shows primary human cardiomyocyte cell viability when cultured in the presence of 1 μM doxorubicin, in the presence of 1 μM doxorubicin and 1 μM bisantrene, in the presence of 1 μM epirubicin, or in the presence of 1 μM epirubicin and 1 μM bisantrene for 72 hours.

FIG. 7 shows primary human cardiomyocyte cell viability when cultured in the presence of approved proteasome inhibitors, or when cultured also in the presence of 1 μM bisantrene at the listed concentrations for 72 hours.

FIG. 8 shows MDA-MB-231 breast cancer cell viability when cultured in the presence of: (A) 0-2 μM bisantrene alone, 0-1 μM doxorubicin alone, 0-2 μM bisantrene in combination with 0-1 μM doxorubicin, with expected additive effect of bisantrene and doxorubicin at the respective concentrations; (B) 0-2 μM bisantrene alone, 0-500 nM doxorubicin alone, 0-2 μM bisantrene in combination with 0-500 nM doxorubicin, with expected additive effect of bisantrene and doxorubicin at the respective concentrations; and (C) 0-1 μM bisantrene alone, 0-1 μM doxorubicin alone, 0-1 μM bisantrene in combination with 0-1 μM doxorubicin, with expected additive effect of bisantrene and doxorubicin at the respective concentrations.

FIG. 9 shows MDA-MB-231 breast cancer cell viability when cultured in the presence of: (A) 0-2 μM bisantrene alone, 0-1 μM epirubicin alone, 0-2 μM bisantrene in combination with 0-1 μM epirubicin, with expected additive effect of bisantrene and epirubicin at the respective concentrations; (B) 0-2 μM bisantrene alone, 0-500 nM epirubicin alone, 0-2 μM bisantrene in combination with 0-500 nM epirubicin, with expected additive effect of bisantrene and epirubicin at the respective concentrations; and (C) 0-1 μM bisantrene alone, 0-1 μM epirubicin alone, 0-1 μM bisantrene in combination with 0-1 μM epirubicin, with expected additive effect of bisantrene and epirubicin at the respective concentrations.

FIG. 10 shows MDA-MB-231 breast cancer cell viability when cultured in the presence of: (A) 0-250 nM bisantrene alone, 0-250 nM bisantrene in the presence of 3.9 nM doxorubicin, 0-250 nM bisantrene in the presence of 7.8 nM doxorubicin, 0-250 nM bisantrene in the presence of 15 nM doxorubicin, 0-250 nM bisantrene in the presence of 31.25 nM doxorubicin, 0-250 nM bisantrene in the presence of 62.5 nM doxorubicin; (B) 0-250 nM bisantrene alone, 0-250 nM bisantrene in the presence of 3.9 nM epirubicin, 0-250 nM bisantrene in the presence of 7.8 nM epirubicin, 0-250 nM bisantrene in the presence of 15 nM epirubicin, 0-250 nM bisantrene in the presence of 31.25 nM epirubicin, 0-250 nM bisantrene in the presence of 62.5 nM epirubicin; and (C) 0-250 nM bisantrene alone, 0-250 nM bisantrene in the presence of 0.625 nM carfilzomib, 0-250 nM bisantrene in the presence of 1.25 nM carfilzomib, 0-250 nM bisantrene in the presence of 2.5 nM carfilzomib, 0-250 nM bisantrene in the presence of 5 nM carfilzomib, 0-250 nM bisantrene in the presence of 10 nM carfilzomib.

FIG. 11 shows primary human cardiomyocyte cell viability when cultured in the presence of 0-10 μM of the FTO inhibitor FB23/2 alone, or when cultured also in the presence of 1 μM doxorubicin or 1 μM carfilzomib for 72 hours.

FIG. 12 shows primary human cardiomyocyte cell viability when cultured in the presence of 0-10 μM of the FTO inhibitor brequinar alone, or when cultured also in the presence of 1 μM doxorubicin or 1 μM carfilzomib after: (A) 24 hours; (B) 48 hours; and (C) 72 hours.

FIG. 13 shows MCF-7 breast cancer cell viability when cultured in the presence of the FTO inhibitor FB23/2 (A) or brequinar (B) when cultured with the FTO inhibitor alone, or when cultured also in the presence of 1 μM doxorubicin for 72 hours.

FIG. 14 shows: (A) Effect of bisantrene on left ventricular shortening in mice, when administered alone or in combination with doxorubicin; and (B) Effect of bisantrene on heart E/A ratio in mice, when administered alone or in combination with doxorubicin. Vehicle alone; 7.33 mg/kg bisantrene once weekly for 4 weeks alone; 5 mg/kg doxorubicin once weekly for 4 weeks alone (N); 5 mg/kg doxorubicin: 3.67 mg/kg bisantrene (Dox: Bis 1:1) once weekly for 4 weeks; 5 mg/kg doxorubicin: 7.33 mg/kg bisantrene (Dox: Bis 1:2) once weekly for 4 weeks; 4 mg/kg doxorubicin: 5.65 mg/kg bisantrene (Dox: Bis 80:20) once weekly for 4 weeks. *: p<0.001, Dox+Vehicle vs. Vehicle at Day 21 & Day 42. #: p<0.05, Dox+Bis 1:1 vs. Dox+Vehicle at Day 21 & Day 42. &: p<0.01 Dox+Bis 1:2 vs. Dox+Vehicle at Day 42 in (A) or p<0.05 Dox+Bis 1:2 vs. Dox+Vehicle at Day 42 in (B).

FIG. 15 shows: (A) Effect of bisantrene on left ventricular diastolic function in mice, when administered alone or in combination with doxorubicin; (B) Effect of bisantrene on left ventricular dilatation in mice, when administered alone or in combination with doxorubicin; and (C) Effect of bisantrene on cardiac output in mice, when administered alone or in combination with doxorubicin. Vehicle alone; 7.33 mg/kg bisantrene once weekly for 4 weeks alone; 5 mg/kg doxorubicin once weekly for 4 weeks alone (0); 5 mg/kg doxorubicin: 3.67 mg/kg bisantrene (Dox: Bis 1:1) once weekly for 4 weeks; 5 mg/kg doxorubicin: 7.33 mg/kg bisantrene (Dox: Bis 1:2) once weekly for 4 weeks; 4 mg/kg doxorubicin: 5.65 mg/kg bisantrene (Dox: Bis 80:20) once weekly for 4 weeks. *: p<0.01, Dox+Vehicle vs. Vehicle at Day 42. #: in (A) and (B) p<0.05, Dox+Bis 1:1 vs. Dox+Vehicle at Day 42; in (C) p<0.01, Dox+Bis 1:1 vs. Dox+Vehicle at Day 42. &: p<0.09 Dox+Bis 1:2 vs. Dox+Vehicle at Day 42 in (A); p<0.19 Dox+Bis 1:2 vs. Dox+Vehicle at Day 42 in (B); p<0.01 Dox+Bis 1:2 vs. Dox+Vehicle at Day 42 in (B).

FIG. 16 shows: (A) Effect of bisantrene on plasma triglyceride levels in mice, when administered alone or in combination with doxorubicin-*: p<0.05, Dox+Vehicle vs. Vehicle at Day 21 & Day 42; #: p<0.05, Dox+Bis 1:1 vs. Dox+Vehicle at Day 21 & Day 42; &: p<0.05 Dox+Bis 1:2 vs. Dox+Vehicle at Day 42; (B) Effect of bisantrene on blood creatine kinase-MB (CK-MB) in mice, when administered alone or in combination with doxorubicin *: p<0.01, Dox+Vehicle vs. Vehicle at Day 21; #: p<0.05, Dox+Bis 1:1 vs. Dox+Vehicle at Day 21 & Day 42; &: p<0.16 Dox+Bis 1:2 vs. Dox+Vehicle at Day 21; and (C) Effect of bisantrene on plasma brain natriuretic peptide (BNP) in mice, when administered alone or in combination with doxorubicin. *: p<0.05, Dox+Vehicle vs. Vehicle at Day 21 & Day 42. #: p<0.05, Dox+Bis 1:1 vs. Dox+Vehicle at Day 21 & Day 42.

FIG. 17 shows: (A) Effect of bisantrene on histologic cardiac fibrosis in mice, when administered alone or in combination with doxorubicin—*: p<0.01, Dox+Vehicle vs. Vehicle at Day 21; #: p<0.05, Dox+Bis 1:1 vs. Dox+Vehicle at Day 21.

### Definitions

As used herein, “treating” means affecting a subject, tissue or cell to obtain a desired pharmacological and/or physiological effect and includes inhibiting a condition, i.e. slowing or arresting its development; or relieving or ameliorating the effects of the condition i.e., cause reversal or regression of the effects of the condition.

As used herein, “preventing” means preventing a condition from occurring in a cell, tissue or subject that may be at risk of having the condition, but does not necessarily mean that condition will not eventually develop, or that a subject will not eventually develop a condition. Preventing includes delaying the onset of a condition in a cell, tissue or subject.

As used herein, “reducing cardiotoxicity” means reducing cardiotoxicity in a subject relative to the cardiotoxicity in a subject not treated to reduce cardiotoxicity as described herein. Reducing the cardiotoxicity may involve, for example, reducing the severity or number of symptoms presenting relative to the severity or number of symptoms presenting in an untreated response.

As used herein, “cardiotoxicity” refers to damage to cardiac tissue, such as damage to cardiomyocytes. Typically, the cardiotoxicity is induced or exacerbated by a cardiotoxic agent.

An agent is “cardioprotective” if it prevents or reduces cardiotoxicity. In the context of bisantrene derivatives and pharmaceutically acceptable salts thereof, it is not expected that all derivatives of bisantrene or salts thereof will be cardioprotective. Rather, where reference is made to a “cardioprotective agent comprising bisantrene or a derivative thereof, or a pharmaceutically acceptable salt of bisantrene or derivative thereof” only derivatives or pharmaceutically acceptable salts thereof that are cardioprotective are contemplated by the present invention.

As used herein, the term “subject” refers to a mammal. The mammal may be a human or a non-human. Examples of non-humans include primate, livestock animal (e.g. sheep, cow, horse, donkey, pig), companion animal (e.g. dog, cat), laboratory test animal (e.g. mouse, rabbit, rat, guinea pig, hamster), captive wild animal (e.g. fox, deer). Typically, the mammal is a human or a non-human primate. More typically, the mammal is a human.

The term “composition” encompasses compositions and formulations comprising the active pharmaceutical ingredient (“bisantrene or a cardioprotective derivative of bisantrene, or a pharmaceutically acceptable salt of bisantrene or cardioprotective derivative of bisantrene”) with excipients or carriers, and also compositions and formulations with a carrier. In pharmaceutical compositions, the excipient or carrier is “pharmaceutically acceptable” meaning that it is not biologically or otherwise undesirable, i.e., the material may be incorporated into a pharmaceutical composition administered to a patient without causing any undesirable biological effects or interacting in a deleterious manner with any of the other components of the composition in which it is contained. Supplementary active ingredients can also be incorporated into the compositions.

By “pharmaceutically acceptable” such as in the recitation of a “pharmaceutically acceptable salt” or a “pharmaceutically acceptable excipient or carrier” is meant herein a material that is not biologically or otherwise undesirable, i.e., the material may be incorporated into a pharmaceutical composition administered to a patient without causing any undesirable biological effects or interacting in a deleterious manner with any of the other components of the composition in which it is contained.

The term “effective amount” or “therapeutically effective amount” refers to the quantity of an active pharmaceutical ingredient that is sufficient to yield a desired therapeutic response. The specific effective amount or therapeutically effective amount will vary with such factors as the particular condition being treated, the age, body weight, general health, physical condition, gender and diet of the subject, the duration of the treatment, the nature of concurrent therapy (if any), and the severity of the particular condition.

As used herein, “carrier” includes any and all solvents, dispersion media, vehicles, coatings, diluents, bulking agents, carrier solutions, suspensions, colloids, and forming and binding agents, any or all of which may include other pharmaceutical excipients as known in the art, including lubricants, antibacterial and antifungal agents, isotonic and absorption delaying agents, buffers, antioxidants, other stabilisers, including physical stabilisers such as thickeners and viscosity enhancers, colouring agents, flavouring and sweetening agents, and the like. The use of such media and agents for pharmaceutical active substances is well known in the art. Except insofar as any conventional media or agent is incompatible with the active ingredient, its use in the therapeutic compositions is contemplated.

As used herein, “administration” or “administer” or “administering” refers to dispensing, applying, or tendering one or more agents to a subject. Administration can be performed using any of a number of methods known in the art. For example, “administering” as used herein is meant via infusion (intravenous administration (i.v.)), parenteral administration. By “parenteral” is meant intravenous, subcutaneous and intramuscular administration.

### DETAILED DESCRIPTION

One aspect provides a method for preventing or reducing drug-induced cardiotoxicity in a subject caused by a cardiotoxic agent, the method comprising administering to the subject an effective amount of bisantrene, a cardioprotective derivative of bisantrene, or a pharmaceutically acceptable salt of bisantrene or cardioprotective derivative of bisantrene.

### Cardiotoxicity

Cardiotoxicity is toxicity to cardiac cells, typically, toxicity to cardiomyocytes. The manifestations are usually chest pain due to myopericarditis and/or palpitations due to sinus tachycardia, paroxysmal nonsustained supraventricular tachycardia and premature atrial and ventricular beats. The electro-cardiogram may reveal nonspecific ST-T changes, left axis deviation and decreased amplitude of QRS complexes. Cardiotoxicity manifests in symptoms which range in severity from overt clinical symptoms such as heart failure, to asymptomatic detectable changes in cardiac function such as electrophysiological dysfunction, contractile dysfunction, and cardiomyocyte apoptosis.

Typically, electrophysiological dysfunction comprises QT prolongation, and contractile dysfunction comprises reduced cardiac ejection fraction (EF) or fractional shortening (FS).

Cardiotoxicity may be measured using a number of approaches.

One current measure for cardiotoxicity is a decrease in left ventricular ejection fraction (LVEF) volume relative to baseline. In this regard, the current standard definition for cardiotoxicity is defined by the Cardiac Review and Evaluation Committee (CREC) on trastuzumab-associated cardiotoxicity and the ESMO Clinical Practice Guideline, and is defined as an echocardiographic left ventricular ejection fraction (LVEF) decrease of more than 10% to less than 50% from baseline. Methods for echocardiographic measurement of LVEF are known in the art, and described in, for example, in Chatterjee, K., Zhang, J., Honbo, N. & Karliner, J. S. Doxorubicin Cardiomyopathy. Cardiology 115, 155-162 (2010).

Another measure of cardiotoxicity is an increase in expression of cardiac marker proteins such as, for example, cardiac troponins. Methods for measuring of cardiac troponin levels are known in the art, and described in, for example, Dong, J. & Chen, H. Cardiotoxicity of Anticancer Therapeutics. Frontiers Cardiovasc Medicine 5, 9 (2018).

Elevated or abnormal expression levels of several biomarkers can be used as indicators for screening and assess the risk factors for future cardiotoxicity complications. Interleukin-6 (IL-6), a cytokine produced by adipose tissues, increases blood pressure and induces inflammation. Overexpression of IL-6 inhibits cell apoptosis, stimulates angiogenesis, and plays a role in drug resistance (Guo Y, Xu F, Lu T, Duan Z, Zhang Z. Interleukin-6 signaling pathway in targeted therapy for cancer. Cancer Treat Rev (2012) 38:904-10). There have also been promising data exploring biomarkers that pertain to type I and type II cardiac events. Increases in brain-type natriuretic peptide (BNP), and N-terminal-pro-BNP have all been linked to drops in LVEF (Jain D, Russell R R, Schwartz R G, Panjrath G S, Aronow W. Cardiac complications of cancer therapy: pathophysiology, identification, prevention, treatment, and future directions. Curr Cardiol Rep (2017) 19:36). Plasma myeloperoxidase also predicts a decrease in cardiac function (Tromp, J., Steggink, L., Veldhuisen, D. V., Gietema, J. & Meer, P. van der. Cardio-Oncology: Progress in Diagnosis and Treatment of Cardiac Dysfunction. Clin Pharmacol Ther 101, 481-490 (2017)). MicroRNA has emerged as a potential marker for early onset of heart failure. miR-1, miR-133b, and miR-146a were all overexpressed after doxorubicin chemotherapy (Cappetta, D. et al. Doxorubicin targets multiple players: A new view of an old problem. Pharmacol Res 127, 4-14 (2018)).

Typically, cardiotoxicity is induced or exacerbated by a cardiotoxic agent. Typically, cardiotoxicity is induced or exacerbated by treatment of a subject with a cardiotoxic agent, usually a therapeutic agent-many chemotherapeutic agents, especially anthracyclines, and a number of others, such as carfilzomib, bortezomib and trastuzumab, are cardiotoxic.

One aspect therefore provides a method for preventing or reducing drug-induced cardiotoxicity induced or exacerbated by a cardiotoxic agent in a subject, the method comprising administering an effective amount of a cardioprotective agent comprising bisantrene or a derivative thereof, or a pharmaceutically acceptable salt of bisantrene or derivative thereof.

### Cardiotoxic Agents

The cardioprotective properties of bisantrene, cardioprotective derivatives of bisantrene, or pharmaceutically acceptable salts thereof make them useful as cardioprotective agents for subjects being treated with a cardiotoxic agent, especially cardiotoxic chemotherapeutic agent. Typically, the cardiotoxic chemotherapeutic agent is for the treatment of cancer.

In one embodiment, the cardiotoxic chemotherapeutic agent is an anthracycline or a pharmaceutically acceptable salt thereof. Examples of anthracyclines include daunorubicin, doxorubicin, epirubicin, idarubicin, mitoxantrone, and valrubicin.

In one embodiment, the anthracycline is doxorubicin.

In one embodiment, the anthracycline is daunorubicin.

In one embodiment, the anthracycline is epirubicin.

In one embodiment, the anthracycline is idarubicin.

In one embodiment, the anthracycline is mitoxantrone.

In one embodiment, the anthracycline is valrubicin.

In one embodiment, there is provided a method for preventing or reducing cardiotoxicity induced or exacerbated by an anthracycline, or a pharmaceutically acceptable salt thereof, in a subject, the method comprising administering an effective amount of a cardioprotective agent comprising bisantrene or a derivative thereof, or a pharmaceutically acceptable salt of bisantrene or derivative thereof. The cardioprotective agent may be administered prior to, simultaneously with, or after, administration of the anthracycline, or a pharmaceutically acceptable salt thereof.

In another embodiment, the cardiotoxic chemotherapeutic agent is a thymidine kinase inhibitor. Examples of tyrosine kinase inhibitors that are cardiotoxic include imatinib mesylate, dasatinib, nilotinib, sunitinib, sorafenib and lapatinib.

In another embodiment, the cardiotoxic chemotherapeutic agent is an antibody-based chemotherapeutic. An example of a cardiotoxic antibody-based chemotherapeutic is trastuzumab (Herceptin).

In another embodiment, the cardiotoxic chemotherapeutic agent is a proteasome inhibitor. All clinically approved proteasome inhibitors have been associated with cardiotoxic effects. One particular example of a cardiotoxic proteasome inhibitor chemotherapeutic is carfilzomib (Kyprolis®), which has been reported to result in cardiotoxic effects in up to 10% of patients. Another example is bortezomib (Velcade®), for which multiple case reports exist suggesting potential cardiotoxicity.

### Bisantrene

Bisantrene is a compound with direct cytotoxic action as well as genomic and immunologic mechanisms of action, including as an inhibitor (IC₅₀ 142 nM) of the fat mass and obesity-associated protein (FTO), an RNA N6-methyladenosine (m6A) demethylase (Su, R., Dong, L., Li, Y., Gao, M., Han, L., Wunderlich, M., et al. (2020). Targeting FTO Suppresses Cancer Stem Cell Maintenance and Immune Evasion. Cancer Cell, 38 (1), 79-96.e11).

The chemical name for bisantrene is 9,10-anthracenedicarboxaldehyde bis [(4,5-dihydro-1H-imidazol-2-yl)hydrazone]. Typically, bisantrene is administered as a pharmaceutically acceptable salt. Typically, the pharmaceutically acceptable salt of bisantrene is bisantrene dihydrochloride.

The structure of bisantrene dihydrochloride is shown in Formula (I)

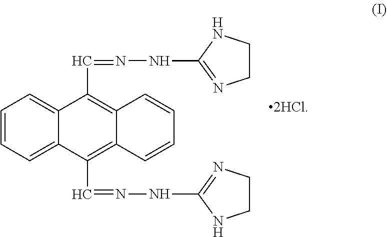 [SMILES: C(=NNC1=NCCN1)c1c2ccccc2c(C=NNC2=NCCN2)c2ccccc12]

Bisantrene dihydrochloride is a tricyclic aromatic compound with the chemical name, 9,10-anthracenedicarboxaldehyde bis [(4, 5-dihydro-1H-imidasol-2-yl) hydrazone] dihydrochloride. The molecular formula of bisantrene hydrochloride is C₂₂H₂₂N₈·2HCl and the molecular weight, 471.4 gmol⁻¹. The alkylimidazole side chains are very basic and, at physiologic pH, are positively charged. This is believed to facilitate electrostatic attractions to negatively charged ribose phosphate groups in DNA.

Bisantrene has a planar structure based around a resonant aromatic ring structure that intercalates within the helices of DNA and disrupts various functions, including replication, presumably due to a strong inhibitory effect on the enzyme topoisomerase II.

Prior to the present disclosure, bisantrene was known to exhibit anti-cancer activity. It was found that bisantrene could kill tumor cells in clonogenic assays and intercalate with DNA, where it inhibits both DNA and RNA synthesis. The primary chemotherapeutic mechanism for bisantrene is its preferential binding to A-T rich regions where it effects changes to supercoiling and initiates strand breaks in association with DNA associated proteins. This results from the inhibition of the enzyme topoisomerase II, which relaxes DNA coiling during replication. It was found that the drug was effective in cancer models using colon 26, Lewis lung, Ridgway osteosarcoma, B16, Lieberman plasma cell, P388 or L1210 cancer cells. Activity in clonogenic assays from 684 patients was seen in breast, small cell lung, large cell lung, squamous cell lung, ovarian, pancreatic, renal, adrenal, head and neck, sarcoma, gastric, lymphoma and melanoma tumor cells, but not in colorectal cancer.

Bisantrene has immunologic properties that might be responsible for some of its anti-cancer activities. Subsequent to treatment with bisantrene, macrophages could be isolated from peritoneal exudate that had cytostatic anti-proliferative functionality in cultures of P815 (mastocytoma) tumor cells. Moreover, the supernatants from bisantrene activated macrophages also had a protective cytostatic effect in the tumor cell cultures. Further work revealed that macrophages activated with bisantrene and adoptively transferred to mice with EL-4 lymphomas more than doubled their median survival time, with 7 of 10 mice in the group being cured. Multiple administrations of activated macrophages were more effective than a single administration. Recent studies have identified that bisantrene suppresses immune checkpoint gene expression and immune evasion via enzymatic inhibition of the FTO RNA demethylase as described in R. Su. et al., “Targeting FTO Suppresses Cancer Stem Cell Maintenance and Immune Evasion”. Cancer Cell, 38 (1), 79-96.e11 (2020).

Bisantrene has also been found to have non-immunologic telomeric effects. Bisantrene binds to DNA at a site called a G-quadruplex, in which 4 guanines are associated by folding. Stabilization of the G-quadruplex can interfere with telomere-telomerase interaction and thus inhibit the activity of telomerase in various ways, including the displacement of telomerase binding proteins. Since the level of topoisomerase II inhibition does not always correlate with cytotoxic efficacy, alternative mechanisms may play a role in the actions of bisantrene.

Bisantrene also possesses other mechanisms which may contribute to its anti-cancer activity, including immunopotentiation. These mechanisms are described in, for example: (i) N. R. West et al., “Tumor-Infiltrating Lymphocytes Predict Response to Anthracycline-Based Chemotherapy in Estrogen-Resistant Breast Cancer,” Breast Canc. Res. 13: R126 (2011), which concludes that the level of tumor-infiltrating lymphocytes is correlated with a response to the administration of anthracycline-based agents; the markers associated with tumor-infiltrating lymphocytes (TIL) include CD19, CD3D, CD48, GZMB, LCK, MS4A1, PRF1, and SELL; (ii) L. Zitvogel et al., “Immunological Aspects of Cancer Chemotherapy,” Nature Rev. Immunol. 8:59-73 (2008), which states that DNA damage, such as that produced by intercalating agents such as bisantrene, induces the expression of NKG2D ligands on tumor cells in an ATM-dependent and CHK1-dependent (but p53-independent) manner; NKG2D is an activating receptor that is involved in tumor immunosurveillance by NK cells, NKT cells, γδ T cells and resting (in mice) and/or activated (in humans) CD8⁺ T cells, and also states that anthracycline-based agents may act as immunostimulators, particularly in combination with IL-12; such agents also promote HMGB1 release and activate T cells; (iii) D. V. Krysko et al., “TLR2 and TLR9 Are Sensors of Apoptosis in a Mouse Model of Doxorubicin-Induced Acute Inflammation,” Cell Death Different. 18:1316-1325 (2011), which states that anthracycline-based antibiotics induce an immunogenic form of apoptosis that has immunostimulatory properties mediated by MyD88, TLR2, and TLR9; (iv) C. Ferraro et al., “Anthracyclines Trigger Apoptosis of Both G0-G1 and Cycling Peripheral Blood Lymphocytes and Induce Massive Deletion of Mature T and B Cells,” Cancer Res. 60:1901-1907 (2000), which stated that anthracyclines induce apoptosis and ceramide production, as well as activate caspase-3 in resting and cycling cells; the apoptosis induced is independent from CD95-L/CD95 and TNF/TNF-R; (v) K. Lee et al., “Anthracycline Chemotherapy Inhibits HIF-1 Transcriptional Activity and Tumor-Induced Mobilization of Circulating Angiogenic Cells,” Proc. Natl. Acad. Sci. USA 106:2353-2358 (2009), which provides another antineoplastic mechanism for anthracycline-based antibiotics, namely inhibition of HIF-1 mediated gene transcription, which, in turn, inhibits transcription of VEGF required for angiogenesis; HIF-1 also activates transcription of genes encoding glucose transporter GLUT1 and hexokinases HK1 and HK2, which are required for the high level of glucose uptake and phosphorylation that is observed in metastatic cancer cells, and pyruvate dehydrogenase kinase 1 (PDK1), which shunts pyruvate away from the mitochondria, thereby increasing lactate production; patients with HIF-la overexpression based on immunohistochemical results were suggested to be good candidates for treatment with anthracycline-based antibiotics; and (iv) R. Su et al., “Targeting FTO Suppresses Cancer Stem Cell Maintenance and Immune Evasion.” Cancer Cell, 38 (1), 79-96.e11 (2020), which identified a new mechanism of action of bisantrene via the inhibition of the FTO enzyme. Inhibition of FTO was found to enhance immunotherapy response via suppression of immune checkpoint gene expression.

### Derivatives of Bisantrene

Derivatives of bisantrene are described in US Patent US20160166546 by Garner et al. Derivatives of bisantrene include, for example, compounds of the following structure:

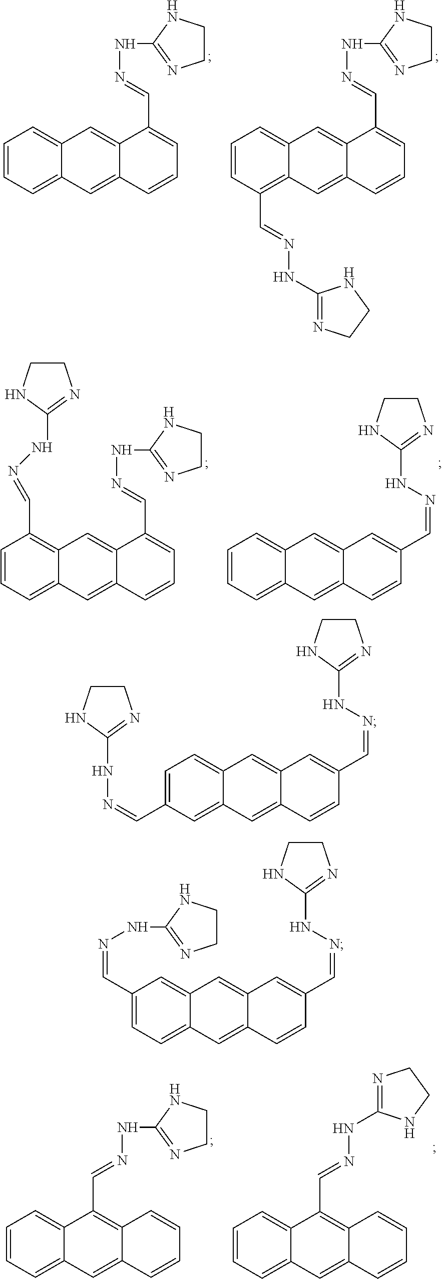

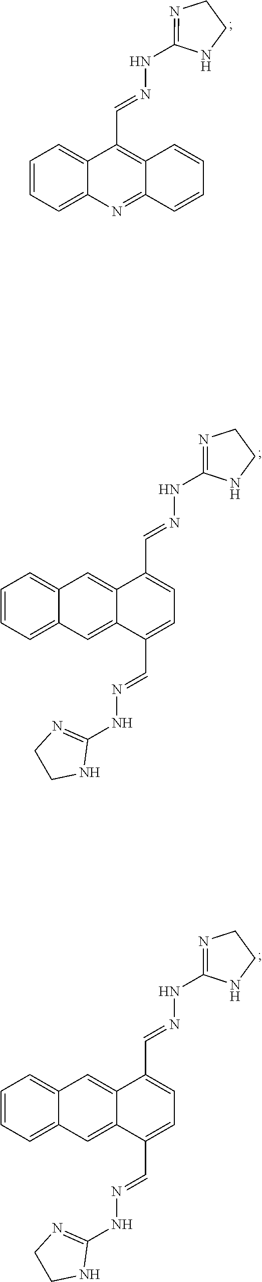 [SMILES: C(=N\NC1=NCCN1)/c1c2ccccc2nc2ccccc12 ; C(=N\NC1=NCCN1)/c1ccc(/C=N/NC2=NCCN2)c2cc3ccccc3cc12 ; C(=N\NC1=NCCN1)/c1ccc(/C=N/NC2=NCCN2)c2cc3ccccc3cc12]

Further examples of derivatives of bisantrene include compounds of the following structure:

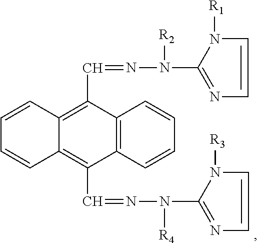

wherein R₁ and R₃ are the same or different and are selected from: hydrogen, C₁-C₆ alkyl, —C(O)—R₅, wherein R₅ is hydrogen, C₁-C₆ alkyl, phenyl, mono-substituted phenyl (wherein the substituent can be ortho, meta, or para and is fluoro, nitro, C₁-C₆ alkyl, C₁-C₃ alkoxy, or cyano), pentafluorophenyl, naphthyl, furanyl,

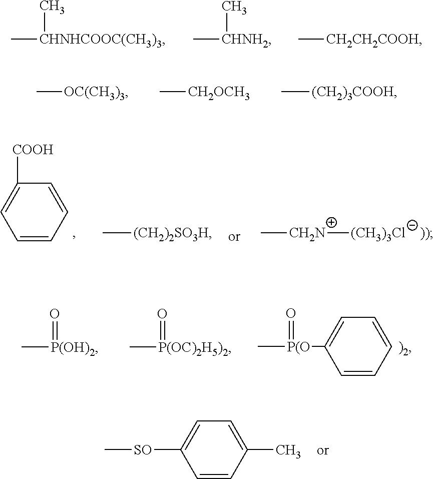

SO₃H;
wherein only one of R₁ and R₃ may be hydrogen or C₁-C₆ alkyl; R₂ and R₄ are the same or different and are selected from: hydrogen, C₁-C₄ alkyl or —C(O)—R₆,
where Re is hydrogen, C₁-C₆ alkyl, phenyl, mono-substituted phenyl (wherein the substituent may be in the ortho, meta, or para position and is fluoro, nitro, C₁-C₆ alkyl, C₁-C₃ alkoxy, or cyano), pentafluorophenyl, naphthyl, furanyl, or —CH₂OCH₃. The compounds can have the schematic structure B(Q)ₙ, wherein B is the residue formed by removal of a hydrogen atom from one or more basic nitrogen atoms of an amine, amidine, guanidine, isourea, isothiourea, or biguanide-containing pharmaceutically active compound, and Q is hydrogen or A, wherein A is

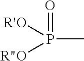

such that R′ and R″ are the same or different and are R where R is C₁-C₆ alkyl, aryl, arylalkyl, heteroalkyl, NC—CH₂CH₂—,

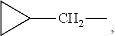

Cl₃C—C₂—, or R₇OCH₂CH₂—, where R₇ is hydrogen or C₁-C₆ alkyl, hydrogen, or a pharmaceutically acceptable cation or R′ and R″ are linked to form a —CH₂CH₂— group or a

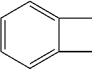

group, and n is an integer representing the number of primary or secondary basic nitrogen atoms in the compound such that at least one Q is A.
Further examples of derivatives of bisantrene include the following compounds:
9,10-bis [(2-hydroxyethyl)iminomethyl]anthracene;
9,10-bis {[2-(-2-hydroxyethylamino)ethyl]iminomethyl}anthracene;
9,10-bis {[2-(-2-hydroxyethylamino)ethyl]iminomethyl}anthracene;
9,10-bis {[2-(morpholin-4-yl)ethyl]iminomethyl}anthracene;
9,10-bis [(2-hydroxyethyl)aminomethyl]anthracene;
9,10-bis {[2-(2-hydroxyethylamino)ethyl]aminomethyl}anthracene tetrahydrochloride;
9,10-bis {[2-(piperazin-1-yl)ethyl]aminomethyl}anthracene hexahydrochloride;
9,10-bis {[2-(morpholin-4-yl)ethyl]aminomethyl}anthracene tetrahydrochloride;
N, N′-bis [2-(dimethylamino)ethyl]-9,10-anthracene-bis(methylamine); and
N, N′-bis(1-ethyl-3-piperidinyl)-9,10-anthracene-bis(methylamine).

Derivatives of bisantrene which are cardioprotective can be readily determined using the methods described herein.

### Methods of Treatment

In one aspect the present invention provides a method of preventing or reducing drug-induced cardiotoxicity in a subject, the method comprising administering to the subject an effective amount of a cardioprotective agent comprising bisantrene or a derivative thereof, or a pharmaceutically acceptable salt of bisantrene or derivative thereof.

The drug-induced cardiotoxicity may be brought about by any cardiotoxic agent a subject has been exposed to, or which has been produced endogenously. However, typically the cardiotoxicity is brought about by exposure of a subject to, or administration to a subject of a cardiotoxic agent, such as a cardiotoxic therapeutic agent, especially cardiotoxic chemotherapeutic agent, such as an anthracycline, a proteasome inhibitor or a tyrosine kinase inhibitor.

Thus, in a specific aspect the present invention provides a method of preventing or reducing cardiotoxicity in a subject being treated with a cardiotoxic chemotherapeutic agent, the method comprising administering to the subject an effective amount of a cardioprotective agent comprising bisantrene or a derivative thereof, or a pharmaceutically acceptable salt of bisantrene or derivative thereof.

In an even more specific aspect the present invention provides a method of treating cancer in a subject, the method comprising administering to the subject:
a. a cardiotoxic chemotherapeutic agent in an amount that is therapeutically effective against the cancer; and
b. a cardioprotective agent comprising bisantrene or a derivative thereof, or a pharmaceutically acceptable salt of bisantrene or derivative thereof, in an amount that is cardioprotective.

The cancer may be any cancer for which treatment with a cardiotoxic chemotherapeutic agent is effective. For example, anthracyclines have been shown to be effective for the treatment of a broad range of cancers including breast cancer, leukemia, lymphoma, stomach cancer, uterine cancer, ovarian cancer, bladder cancer, lung cancer, melanoma, and myeloma. In one embodiment, the cancer is selected from the group consisting of bladder cancer, bone cancer, brain tumor, breast cancer, esophageal cancer, colorectal cancer, leukemia, liver cancer, lung cancer, lymphoma, myeloma, ovarian cancer, prostate cancer, sarcoma, stomach cancer, and thyroid cancer. According to one embodiment the cancer is clear cell renal carcinoma. According to another embodiment the cancer is melanoma. According to another embodiment the cancer is multiple myeloma. According to another embodiment the cancer is acute myeloid leukemia.

The pharmaceutical compositions and medicaments of the present invention may be administered to a subject by standard enteral or parenteral routes, including, but not limited to, injection (such as intravenous, subcutaneous, intramuscular, bolus, etc.), or by, for example, topical, oral, sublingual, nasal, pulmonary, otic, rectal or vaginal administration routes. In some embodiments, pharmaceutical compositions according to the invention may be administered to a subject by themselves or in combination with other pharmaceutical composition(s). In the latter case, the administration may be simultaneous or sequential, or administration of the pharmaceutical composition(s) may be independent of one another.

In certain embodiments bisantrene is administered intravenously, either centrally or peripherally, including by intramuscular, subcutaneous and/or intradermal injection.

In general, the pharmaceutical compositions and medicaments of the present invention can be administered in a manner compatible with the route of administration and physical characteristics of the subject (including health status) and in such a way that the desired effect(s) are induced (i.e. therapeutically effective and/or preventative). For example, the appropriate dosage may depend on a variety of factors including, but not limited to, a subject's physical characteristics (e.g. age, weight, sex), whether the composition or medicament is being used as single agent, the progression (i.e. pathological state) of the disease or disorder being treated, and other factors readily apparent to those of ordinary skill in the art.

Suitable dosages, dosage frequencies, dosage durations, and routes of administration for chemotherapeutic agents are known in the art. Bisantrene, cardioprotective derivatives of bisantrene, or pharmaceutically acceptable salts of bisantrene or a cardioprotective derivative of bisantrene can either be administered in the same pharmaceutical composition as a cardiotoxic chemotherapeutic agent, in a separate composition as, but simultaneously with the cardiotoxic chemotherapeutic agent, or at a different time. If the bisantrene, cardioprotective derivative of bisantrene, or pharmaceutically acceptable salt of bisantrene or a cardioprotective derivative of bisantrene, is administered at a different time than the cardiotoxic chemotherapeutic agent, it can either be administered before or after the cardiotoxic chemotherapeutic agent and/or at different timings, being administered according to different timing and/or frequency regimes. One of ordinary skill in the art can determine a suitable schedule for administration based on variables such as the age, weight, and sex of the subject, the susceptibility of the subject to cardiotoxicity, genetic markers, the dose of cardiotoxic agent, the subjects history with prior cardiotoxic agents, and pharmacokinetic parameters such as heart function.

The methods and compositions provided herein enable a subject to receive a therapy more frequently without having the dosage regimen significantly altered by the risk of cardiotoxicity. The daily dose of a cardiotoxic chemotherapeutic agent and bisantrene, cardioprotective derivative of bisantrene, or pharmaceutically acceptable salt of bisantrene or a cardioprotective derivative of bisantrene, may be administered to a subject in one or more doses per day. In some cases, the daily dose of the chemotherapeutic agent may be administered together with bisantrene, cardioprotective derivative of bisantrene, or pharmaceutically acceptable salt of bisantrene or a cardioprotective derivative of bisantrene, in a single dose.

The pharmaceutical compositions described herein may be administered to a patient one or more times per day. In some cases, the pharmaceutical composition may be administered to a patient once per day. In some cases, the pharmaceutical composition may be administered to a patient at least 2 times, 3 times, 4 times 5 times, or 6 times per day. For example, a pharmaceutical composition may be administered to a patient 3 times per day.

In methods described herein, suitable dosages of bisantrene (or a cardioprotective derivative of bisantrene) can be determined by one of ordinary skill in the art. The selected dosage level depends upon a variety of pharmacokinetic factors including the amount of cardiotoxic chemotherapeutic agent being administered, the route of administration, the time of administration, the rate of excretion of the particular compound being employed, the severity of the condition, other health considerations affecting the subject, and the status of liver and kidney function of the subject. It also depends on the duration of the treatment, other drugs, compounds and/or materials used in combination with the particular therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20th ed., 2000, and Gilman et al., (Eds), (1990), “Goodman And Gilman's: The Pharmacological Bases of Therapeutics”, Pergamon Press. Optimal dosages for a given set of conditions can be ascertained by those skilled in the art using conventional dosage-determination tests in view of the experimental data for an agent.

According to certain embodiments, administration of bisantrene is performed at a dosage of from about 0.1 mg/m²/day to about 100 mg/m²/day, such as from about 0.2 mg/m²/day to about 50 mg/m²/day, from about 0.5 mg/m²/day to about 20 mg/m²/day, from about 1.0 mg/m²/day to about 10 mg/m²/day, from about 1.0 mg/m²/day to about 8 mg/m²/day, about 1 mg/m²/day, about 2 mg/m²/day, about 3 mg/m²/day, about 4 mg/m²/day, about 5 mg/m²/day, about 6 mg/m²/day, about 7 mg/m²/day, or about 10 mg/m²/day. In some embodiments, bisantrene is administered daily or weekly, once every two weeks, once every three weeks, once every four weeks, over a period of, for example, 1 day, 2 days, 3 days, 4 days, 5 days, 6 days, 7 days, 8 days, 9 days, 10 days, 11 days, 12 days, 13 days, 14 days, 21 days, 28 days, 35 days, 42 days, 49 days, 56 days or 63 days. In certain embodiments, bisantrene is administered once or multiple times over a period of 28 days, optionally once or multiple times daily or weekly, once every two weeks, once every three weeks, once every four weeks, at a dosage of, for example, about 20-100 mg/m^{2/28} days. For example, in certain embodiments, bisantrene may be administered to a patient once weekly over a period of four weeks, with a dosing regime of from about 0.5 mg/m²/week to about 50 mg/m²/week, from about 1 mg/m²/week to about 40 mg/m²/week, from about 2 mg/m²/week to about 30 mg/m²/week, from about 5 mg/m²/week to about 25 mg/m²/week, about 5 mg/m²/week, about 10 mg/m²/week, about 15 mg/m²/week, about 20 mg/m²/week, or about 25 mg/m²/week. Administration of pharmaceutically acceptable salts of bisantrene or cardioprotective derivatives or pharmaceutically acceptable salts thereof may be performed at similar dosage rates, adjusted for molar equivalence.

According to certain embodiments, and as a result of the at least additive, if not synergistic biologic activity observed when the cardioprotective agent is administered with the cardiotoxic agent, a therapeutic outcome may be achieved more effectively for a given dose rate for that cardiotoxic agent. This biologic activity of the cardioprotective agent may also allow for use, or expanded use of cardiotoxic therapeutic agents, such as proteasome inhibitors, such as carfilzomib or bortezomib, which cannot be safely administered to patients, or their administration to patients is limited due to their cardiotoxicity.

Alternatively, when administered in combination with cardioprotective agent, the cardiotoxic agent may be administered to a patient at a lower dosage than it would normally be administered, over a longer period while maintaining a comparable ongoing therapeutic outcome. This is particularly important for cardiotoxic agents with a lifetime cumulative dose limit, like many anthracyclines. Thus, in certain embodiments of methods according to the present invention, when administered in combination with a cardioprotective agent, the cardiotoxic agent may be administered at a rate which is significantly lower than the recommended dosage rate for that cardiotoxic agent when administered alone, such as at a rate which is at least 10%, 20%, 30%, 40%, 50%, 60% or 70% lower than the dosage rate for that cardiotoxic agent when administered alone. For example, typical dosage rates for doxorubicin may be about 50-75 mg/m²/28 days, but when co-administered with bisantrene to a patient undergoing cancer treatment by a method according to the present invention, the doxorubicin may be administered at a dosage of from about 10 mg/m²/28 days to about 60 mg/m²/28 days, such as from about 15 mg/m²/28 days to about 50 mg/m²/28 days, from about 20 mg/m²/28 days to about 50 mg/m²/28 days, or from about 25 mg/m²/28 days to about 45 mg/m²/28 days, with bisantrene being administered at a similar dose rate, so that, for example, dose rates of bisantrene of about 30-40 mg/m²/28 days co-administered with an anthracycline such as doxorubicin, daunorubicin or epirubicin at dose rates of about 30-40 mg/m²/28 days are contemplated for cancer treatment methods according to the present invention.

In certain embodiments of methods of treatment according to the present invention, the dose of cardiotoxic agent and cardioprotective agent may be in a molar ratio of from about 1:10 to about 10:1, such as from about 1:5 to about 5:1, from about 1:4 to 4:1, from about 1:3 to 3:1, from about 1:2 to 2:1, from about 1:1.5 to 1.5:1, or about 1:1. In alternative embodiments, the dose of cardiotoxic agent and dose of cardioprotective agent are independent. For example, where the cardioprotective agent is bisantrene, it may be administered to a patient at a rate as discussed above irrespective of the dosage regime of the cardiotoxic agent, and therefore, for example, once weekly over a period of four weeks, with a dosing regime of from about 0.5 mg/m²/week to about 50 mg/m²/week, from about 1 mg/m²/week to about 40 mg/m²/week, from about 2 mg/m²/week to about 30 mg/m²/week, from about 5 mg/m²/week to about 25 mg/m²/week, about 5 mg/m²/week, about 10 mg/m²/week, about 15 mg/m²/week, about 20 mg/m²/week, or about 25 mg/m²/week, irrespective of the timing or dosage rates of the cardioprotective agent.

### Compositions

The cardioprotective agent is typically administered in a pharmaceutical composition.

The cardioprotective agent may be administered to a subject separately to any other therapeutic agent, or may be administered in combination with a cardiotoxic agent, such as a cardiotoxic chemotherapeutic drug. Thus, according to one embodiment a pharmaceutical composition according to the present invention may comprise a cardioprotective agent comprising bisantrene or a derivative thereof, or a pharmaceutically acceptable salt of bisantrene or derivative thereof. According to another embodiment of the invention, the composition may also comprise a cardiotoxic agent, such as a cardiotoxic chemotherapeutic agent.

In one embodiment, the cardiotoxic chemotherapeutic agent is an anthracycline or a pharmaceutically acceptable salt thereof.

In one embodiment, the anthracycline is selected from daunorubicin, doxorubicin, epirubicin, idarubicin, mitoxantrone, and valrubicin.

In another embodiment, the cardiotoxic chemotherapeutic agent is a proteasome inhibitor, such as carfilzomib or bortezomib.

In other embodiment, the cardiotoxic chemotherapeutic agent is a tyrosine kinase inhibitor.

In other embodiment, the cardiotoxic chemotherapeutic agent is a monoclonal antibody, such as trastuzumab.

Compositions according to the present invention may be administered by any route, and in a form suitable for that route, as known in the art. Thus, compositions according to the present invention may be adapted for administration by enteral or parenteral routes, including by injection (such as intravenous, subcutaneous, intramuscular, bolus, etc.), or by, for example, topical, oral, sublingual, nasal, pulmonary, otic, rectal or vaginal administration routes.

Typically, the pharmaceutical compositions described herein include at least one pharmaceutically acceptable carrier or excipient and/or diluents. For preparing the pharmaceutical compositions and medicaments, inert, pharmaceutically acceptable carriers can be either solid or liquid. Liquid form preparations include solutions, suspensions and emulsions, for example water or water-propylene glycol solutions for parenteral injection. Also included are solid form preparations, such as tablets, or amorphous or crystalline powders, including lyophilized preparations, that are intended to be converted, shortly before use, to liquid form preparations for either oral or injection administration. Such liquid forms include solutions, suspensions and emulsions. Examples of pharmaceutically acceptable carriers and methods of manufacture for various compositions may be found in, for example, “Remington: The Science and Practice of Pharmacy”, Mack Publishing Co., 20th ed., 2000, and Gilman et al., (Eds), (1990), “Goodman And Gilman's: The Pharmacological Bases of Therapeutics”, Pergamon Press.

Pharmaceutically acceptable carriers and excipients include:
(i) a liquid carrier;
(ii) an isotonic agent;
(iii) a wetting or emulsifying agent;
(iv) a preservative;
(v) a buffer;
(vi) an acidifying agent;
(vii) an antioxidant;
(viii) an alkalinizing agent;
(ix) a carrying agent;
(x) a chelating agent;
(xi) a coloring agent;
(xii) a complexing agent;
(xiii) a solvent;
(xiv) a suspending and/or viscosity-increasing agent;
(xv) an oil;
(xvi) a penetration enhancer;
(xvii) a polymer;
(xviii) a stiffening agent;
(xix) a protein;
(xx) a carbohydrate;
(xxi) a bulking agent; and
(xxii) a lubricating agent.

Other pharmaceutically acceptable carriers and excipients known in the art may be used.

The carriers, diluents, excipients and adjuvants must be “acceptable” in terms of being compatible with the other ingredients of the composition or medicament, and are generally not deleterious to the subject thereof. Non-limiting examples of pharmaceutically acceptable carriers or diluents are demineralised or distilled water; saline solution; vegetable-based oils such as peanut oil, safflower oil, olive oil, cottonseed oil, maize oil; sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxylpropylmethylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from about 10% to about 99.9% by weight of the composition, vaccine or medicament.

For administration as an injectable solution or suspension, non-toxic parenterally acceptable diluents or carriers can include, Ringer's solution, isotonic saline, phosphate buffered saline, ethanol and 1,2 propylene glycol. Methods for preparing parenterally administrable pharmaceutical compositions and medicaments are apparent to those of ordinary skill in the art, and are described in more detail in, for example, “Remington: The Science and Practice of Pharmacy”, Mack Publishing Co., 20th ed., 2000, and Gilman et al., (Eds), (1990), “Goodman And Gilman's: The Pharmacological Bases of Therapeutics”, Pergamon Press.

For oral administration, some examples of suitable carriers, diluents, excipients and adjuvants include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl stearate which delay disintegration.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate,-stearate or -laurate and the like.

In certain embodiments, the pharmaceutical composition may comprise a liposome. A liposomal formulation suitable for bisantrene or a cardioprotective derivative thereof comprises small unilamellar or multilamellar liposomes of size range between 0.01 and 100 μM, and between about 50-95% liposome-entrapped bisantrene, composed of hydrogenated soy phosphatidylcholine, distearoyl phosphatidylglycerol, and cholesterol of natural or synthetic origin lipids, in aqueous solution which can be reconstituted from a lyophilized form to an injectable liposome suspension. The composition is prepared by reconstituting a lyophilized bisantrene/liposome composition to a liposome concentrate, then diluting the concentrate for parenteral administration for the treatment of melanoma.

In yet another embodiment, the pharmaceutical composition may comprise a complex with a beta-cyclodextrin. A liposomal formulation suitable for bisantrene, a cardioprotective derivative of bisantrene, or a pharmaceutically acceptable salt of bisantrene or a cardioprotective derivative of bisantrene, comprises a complex formed in aqueous solution which may be reconstituted from a lyophilized form to an injectable suspension. One such composition is prepared by reconstituting a lyophilized bisantrene/beta-cyclodextrin composition to a concentrate, then diluting the concentrate for parenteral administration. Beta-cyclodextrin complexes and methods for preparing such complexes are known in the art and are described in., for example, WO 2019/073296 by Rothman.

Various formulations suitable for use in the administration of bisantrene, a cardioprotective derivative of bisantrene, or a pharmaceutically acceptable salt of bisantrene or cardioprotective derivative of bisantrene, are known in the art. U.S. Pat. No. 4,784,845 to Desai et al. discloses a composition for delivery of a hydrophobic drug (i.e., bisantrene or a cardioprotective derivative thereof) comprising: (i) the hydrophobic drug; (ii) an oleaginous vehicle or oil phase that is substantially free of butylated hydroxyanisole (BHA) or butylated hydroxytoluene (BHT); (iii) a co-surfactant or emulsifier; (iv) a co-surfactant or auxiliary emulsifier; and (v) benzyl alcohol as a co-solvent. U.S. Pat. No. 4,816,247 by Desai et al. discloses a composition for delivery by intravenous, intramuscular, or intraarticular routes of hydrophobic drugs (such as bisantrene or a cardioprotective derivative or analog thereof) comprising: (i) the hydrophobic drug; (ii) a pharmaceutically acceptable oleaginous vehicle or oil selected from the group consisting of: (a) naturally occurring vegetable oils and (b) semisynthetic mono-, di-, and triglycerides, wherein the oleaginous vehicle or oil is free of BHT or BHA; (iii) a surfactant or emulsifier; (iv) a co-surfactant or emulsifier; (v) an ion-pair former selected from C₆-C₂₀ saturated or unsaturated aliphatic acids when the hydrophobic drug is basic and a pharmaceutically acceptable aromatic amine when the hydrophobic drug is acidic; and (vi) water.

According to certain embodiments, compositions according to the present invention may be adapted for administration of bisantrene at a dosage of from about 0.1 mg/m²/day to about 100 mg/m²/day, such as from about 0.2 mg/m²/day to about 50 mg/m²/day, from about 0.5 mg/m²/day to about 20 mg/m²/day, from about 1.0 mg/m²/day to about 10 mg/m²/day, from about 1.0 mg/m²/day to about 8 mg/m²/day, about 1 mg/m²/day, about 2 mg/m²/day, about 3 mg/m²/day, about 4 mg/m²/day, about 5 mg/m²/day, about 6 mg/m²/day, about 7 mg/m²/day, or about 10 mg/m²/day. In some embodiments, compositions according to the present invention may be adapted for administration of bisantrene once or multiple times daily or weekly, once every two weeks, once every three weeks, once every four weeks, over a period of, for example, 1 day, 2 days, 3 days, 4 days, 5 days, 6 days, 7 days, 8 days, 9 days, 10 days, 11 days, 12 days, 13 days, 14 days, 21 days, 28 days, 35 days, 42 days, 49 days, 56 days or 63 days. In certain embodiments, compositions according to the present invention may be adapted for administration of bisantrene over a period of 28 days, optionally once or multiple times daily or weekly, once every two weeks, once every three weeks, once every four weeks, at a dosage of about 20-50 mg/m²/28 days. Compositions according to the present invention comprising pharmaceutically acceptable salts of bisantrene or cardioprotective derivatives or pharmaceutically acceptable salts thereof may be adapted to deliver similar dosage rates, adjusted for molar equivalence.

In certain embodiments, compositions according to the present invention comprise both a cardioprotective agent and a cardiotoxic therapeutic agent, where the composition is adapted to deliver the cardioprotective agent at a dosage as described above, as well as the cardiotoxic therapeutic agent at a desired dosage rate according to recommended advice for that cardiotoxic agent or according to the particular protocol designed for the treatment regime being adopted.

In certain embodiments of compositions according to the present invention, the dose of cardiotoxic agent and cardioprotective agent may be in a molar ratio of from about 1:10 to about 10:1, such as from about 1:5 to about 5:1, from about 1:4 to 4:1, from about 1:3 to 3:1, from about 1:2 to 2:1, from about 1:1.5 to 1.5:1, or about 1:1. Where the cardioprotective agent is bisantrene and the cardiotoxic agent is an anthracycline, a composition according to the present invention may, for example, be adapted to deliver the bisantrene at a dosage rate of 20-60 mg/m²/28 days and anthracycline at a dosage rate of about 20-60 mg/m²/28 days. Carfilzomib, on the other hand, has significantly greater specific activity compared to doxorubicin, and will need to be present in the composition at a significantly lower ratio compared to bisantrene or other cardioprotective agent.

### Kits

The present invention also provides kits for reducing, preventing or eliminating drug-induced cardiotoxicity in a subject.

One aspect provides a kit for preventing or reducing cardiotoxicity in a subject, comprising bisantrene, a cardioprotective derivative of a cardioprotective agent comprising bisantrene or a derivative thereof, or a pharmaceutically acceptable salt of bisantrene or derivative thereof.

In one embodiment, the kit further comprises a cardiotoxic agent.

In one embodiment, the cardiotoxic agent is a cardiotoxic chemotherapeutic agent.

In one embodiment, the cardiotoxic chemotherapeutic agent is an anthracycline or a pharmaceutically acceptable salt thereof.

In one embodiment, the anthracycline is selected from daunorubicin, doxorubicin, epirubicin, idarubicin, mitoxantrone, and valrubicin.

In one embodiment, the anthracycline is doxorubicin, daunorubicin or epirubicin.

In another embodiment the cardiotoxic chemotherapeutic agent is a tyrosine kinase inhibitor.

In another embodiment the cardiotoxic chemotherapeutic agent is a monoclonal antibody such as, for example, trastuzumab.

In another embodiment the cardiotoxic chemotherapeutic agent is a proteasome inhibitor such as, for example, carfilzomib or bortezomib.

In some cases, the kit may also comprise vials, tubes, needles, packaging, or other materials.

Kits with unit doses of one or more of the compounds described herein, usually in injectable doses, are provided. Such kits may include a container containing the unit dose, an informational package insert describing the use and attendant benefits of the drugs in treating the disease, and optionally an appliance or device for delivery of the composition.

The kit may further comprise any device suitable for administration of the composition. For example, a kit may comprise a needle suitable for intravenous administration.

In some cases, kits may be provided with instructions. The instructions may be provided in the kit or they may be accessed electronically. The instructions may provide information on how to use the compositions of the present disclosure. The instructions may further provide information on how to use the devices of the present disclosure. The instructions may provide information on how to perform the methods of the disclosure. In some cases, the instructions may provide dosing information. The instructions may provide drug information such as the mechanism of action, the formulation of the drug, adverse risks, contraindications, and the like. In some cases, the kit is purchased by a physician or health care provider for administration at a clinic or hospital. In some cases, the kit is purchased by a laboratory and used for screening candidate compounds.

Of course, any material used in preparing the pharmaceutical compositions described herein should ideally be pharmaceutically pure and substantially non-toxic in the amounts employed.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

All publications mentioned in this specification are herein incorporated by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In order to exemplify the nature of the present invention such that it may be more clearly understood, the following non-limiting examples are provided.

### Examples

### Example 1—Materials and Methods

### Primary Human Cardiomyocyte Cultures

Primary Human Cardiac Myocytes (HCMs) were purchased from PromoCell® (Heidelberg, Germany) and cultured according to manufacturer's recommendations using PromoCell® Myocyte growth media (5% [v/v] foetal calf serum, 0.5 ng/ml recombinant human epidermal growth factor, 2 ng/ml recombinant human basic fibroblast growth factor, 5 μg/ml recombinant human insulin) supplemented with 1% [v/v] Penicillin-Streptomycin [Sigma-Aldrich]). Briefly, 1×10⁶ HCMs were seeded and grown in Corning® T75 flasks (Sigma-Aldrich, New South Wales, Australia) in an incubator at 37° C. under 5% CO₂ conditions. At 80-90% confluency, HCMs were gently detached using the DetachKit (PromoCell®), collected into Corning® 50 mL centrifuge tubes (Sigma-Aldrich), and centrifuged at 300×g for 10 mins at room temperature. The resulting supernatant were discarded and HCM pellet was resuspended with 5-10 mL of pre-warmed Myocyte Growth Medium. Total viable cells were enumerated with Invitrogen™ Countess Cell Counter (Thermo Fisher Scientific, New South Wales, Australia) and 1×10⁵ live HCMs were seeded in triplicate into Corning® 96-well white polystyrene microplate (Sigma). Seeded HCMs were incubated in an incubator at 37° C. under 5% CO₂ conditions overnight prior to bisantrene treatments (Day −1).

### MCF7 Breast Cancer Cells

Human breast adenocarcinoma (MCF7) cells were purchased from In Vitro Technologies (Victoria, Australia) and cultured according to manufacturer's recommendations using MCF7 growth media, Low glucose Dulbecco's Modified Eagle's medium (DMEM; Sigma-Aldrich, Australia) supplemented with 10% (v/v) foetal calf serum (Sigma-Aldrich) and 1% (v/v) Penicillin-Streptomycin (Sigma-Aldrich). Briefly, 1×10⁶ MCF7 were seeded and grown in Corning® T75 flasks (Sigma-Aldrich) in an incubator at 37° C. under 5% CO₂ conditions. At 80-90% confluency, MCF7 were washed with Dulbecco's Phosphate Buffered Saline (Sigma-Aldrich) for 30s at room temperature and followed by incubation with Trypsin-EDTA (Sigma-Aldrich) for 5 mins at 37° C. under 5% CO₂ conditions. Trypsin solution was then neutralised with 1:1 volume of MCF7 growth media. The cell suspensions were then collected into Corning® 50 mL centrifuge tubes (Sigma-Aldrich) and centrifuged at 300×g for 10 mins at room temperature. The resulting supernatants were discarded and MCF7 pellets were resuspended with 5-10 mL of pre-warmed MCF7 growth media. Total viable cells were enumerated with Invitrogen™ Countess Cell Counter (Thermo Fisher Scientific, New South Wales, Australia) and 9×10⁴ live MCF7 were seeded in triplicate into Corning® 96-well white polystyrene microplate (Sigma). Seeded HCMs were incubated in an incubator at 37° C. under 5% CO₂ conditions overnight prior to bisantrene treatments (Day −1).

### MDA-MB-231 Breast Cancer Cells

Human breast cancer cell line NDA-MB-231 was cultured in a humidified chamber at 37° C. with 5% CO₂ in DMEM supplemented with 10% foetal bovine serum (FBS), 2 mM L-glutamine and 2% HEPES. Cells were maintained adherent.

### H929 Multiple Myeloma Cells

Human multiple myeloma NCI-H929 cells were cultured using RPMI-1640 growth media (Sigma-Aldrich), supplemented with 2 mM L-glutamine (Thermo Fisher Scientific), 10% (v/v) foetal calf serum (Sigma-Aldrich) and 0.05 mM 2-mercaptoethanol (Sigma-Aldrich). Briefly, 0.5×10⁶ H929 were seeded and grown in Corning® T75 flasks (Sigma-Aldrich) in an incubator at 37° C. under 5% CO₂ conditions. At 80-90% confluency, the cell suspension was then collected into Corning® 50 mL centrifuge tubes (Sigma-Aldrich) and centrifuged at 300×g for 10 mins at room temperature. The resulting supernatant were discarded and H929 pellet was resuspended with 5-10 mL of pre-warmed H929 growth media. Total viable cells were enumerated with Invitrogen™ Countess Cell Counter (Thermo Fisher Scientific, New South Wales, Australia) and 1×10⁴ live H929 were seeded in triplicate into Corning® 96-well white polystyrene microplate (Sigma). Seeded H929 were incubated in an incubator at 37° C. under 5% CO₂ conditions overnight prior to bisantrene treatments (Day −1).

### Cell Treatments

To evaluate the therapeutic potential of Bisantrene, HCMs and MCF7 cells were treated with Bisantrene in combination with commercially available anti-cancer agents doxorubicin hydrochloride (optionally in combination with 500 nM trastuzumab (Tz; MedChemExpress, NJ, USA), daunorubicin hydrochloride, epirubicin hydrochloride (Dox, Dauno and Epi; Sigma-Aldrich), carfilzomib, or bortezomib (CFZ and BTZ; MCER). Bisantrene was either provided by RACE Oncology Ltd (Sydney, New South Wales, Australia) or procured from MedChemExpress (MCER, New Jersey, USA). Both bisantrene, CFZ and BTZ were dissolved in dimethyl sulfoxide (DMSO; Sigma-Aldrich) to make a stock concentration of 500 μM and 20 mM respectively. Dox, Dauno and Epi, were dissolved in sterile water to make a stock concentration of 20 mM, respectively, while Tz was dissolved in sterile PBS to a stock concentration of 100 μM. A series of bisantrene concentrations were made in HCMs (0.5% v/v serum), MCF7 (10% v/v serum) or H929 growth media. The concentrations were as follows 0 nM, 15.62 nM, 62.5 nM, 250 nM, 500 nM, 1 μM, 5 μM, and 10 μM. Dox, Epi or CFZ were then diluted in each of the Bisantrene concentrations to a final concentration of 1 μM. On Day 0, spent media were removed from each well, 200 μL of bisantrene treatments (with or without anti-cancer agents) were added accordingly, and cells incubated at 37° C. under 5% CO₂ overnight. Vehicle controls were incubated with media supplemented with either sterile water or DMSO. After 24 hours, spent media were removed and replaced with 200 μL of fresh bisantrene treatments. This was repeated at 48 hours and 72 hours. Cell viability was then assessed at 24, 48, and 72 or only 72 hours-post bisantrene treatments using the CellTiter-Glo® Luminescent Cell Viability Assay (Promega, New South Wales, Australia) according to manufacturer's instruction and luminescence recorded with the Cytation™ 3 Cell Imaging Multi-Mode Reader (BioTek Instruments, Vermont, USA). Similar experiments using the FTO inhibitors FB23/2 and brequinar instead of bisantrene were also carried out. Both FB23/2 and brequinar (MedChemExpress) were dissolved in dimethyl sulfoxide (DMSO; Sigma-Aldrich) to make a stock concentration of 40 mM and 20 mM respectively.

### Example 2—Bisantrene Reduces Toxicity of Cardiotoxic Agents to Cardiomyocytes

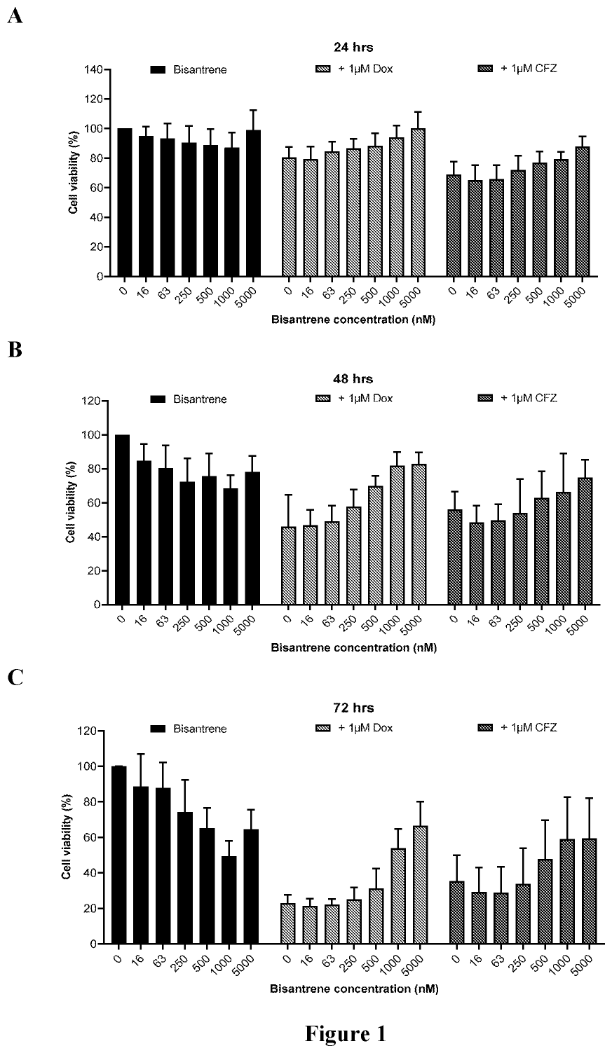

As shown in FIG. 1, doxorubicin and carfilzomib both showed progressive toxicity (measured as loss in cell viability) to cardiomyocytes over time (24 hrs, 48 hrs and 72 hrs), as expected. However, and surprisingly, as also shown in FIG. 1 while bisantrene showed mild progressive toxicity to cardiomyocytes over time (24 hrs, 48 hrs and 72 hrs), it also showed concentration-dependent protection of cardiomyocytes against the progressive toxicity of Dox and CFZ to cardiomyocytes.

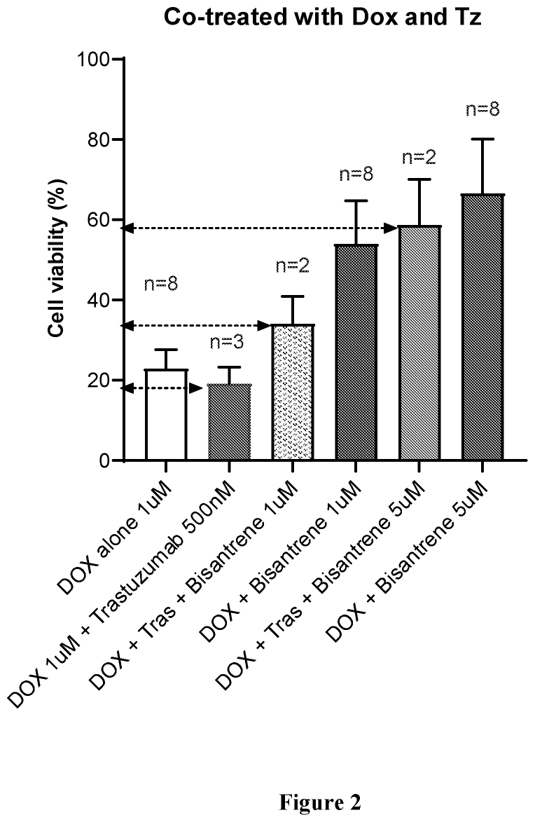

Bisantrene also reduced the combined toxicity of doxorubicin and trastuzumab (the latter also known to cause cardiotoxicity when administered alone) when cardiomyocytes were cultured in the presence of doxorubicin and trastuzumab. As shown in FIG. 2, culturing of cardiomyocytes in the presence of trastuzumab and doxorubicin resulted in reduced viability of the cardiomyocytes as compared to culturing in the presence of doxorubicin alone. Increasing concentrations of bisantrene in the culture media, whether comprising doxorubicin alone or doxorubicin and trastuzumab, resulted in reduced loss of viability. While viability of cardiomyocytes in the presence of trastuzumab as well as doxorubicin was lower than in the presence of doxorubicin alone, the relative difference in viabilities became less significant when 5 μM bisantrene was included in the culture medium compared to when 1 μM bisantrene was included in the culture medium.

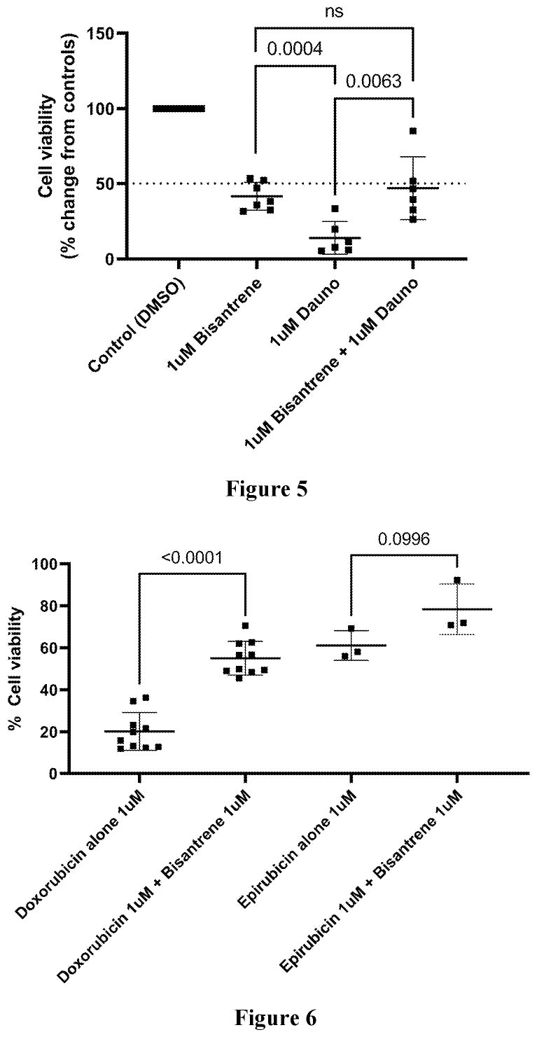

FIG. 5 demonstrates that the cardiotoxicity rescue effects of bisantrene on cardiomyocytes extended to the anthracycline, daunorubicin when both agents were administered at 1 μM. Similarly, a trend toward statistical significance was observed for coadministration of epirubicin and bisantrene at 1 μM, presented in FIG. 6. These observations support that the cardioprotective protective effects of bisantrene are general across the anthracycline class of chemotherapeutic agents.

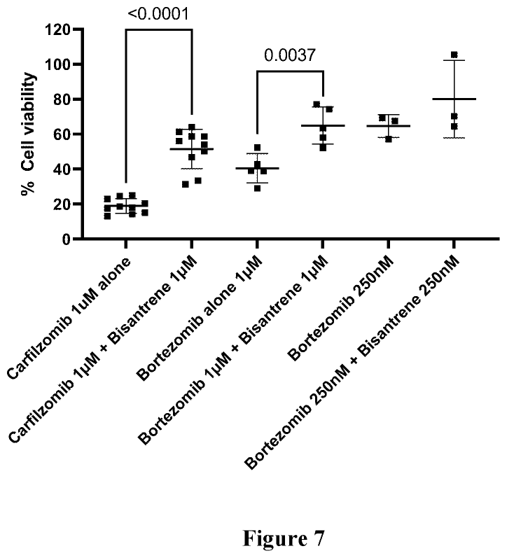

FIG. 7 further illustrates that cardioprotective effects of bisantrene are not specific to the anthracycline class, with significant rescue of cardiomyocyte toxicity observed for both the irreversible proteasome inhibitor, carfilzomib and the reversible inhibitor bortezomib when each agent was coadministered with bisantrene.

### Example 3—Bisantrene is Toxic to Cancer Cells and does not Protect them Against Toxicity of Cardiotoxic Agents

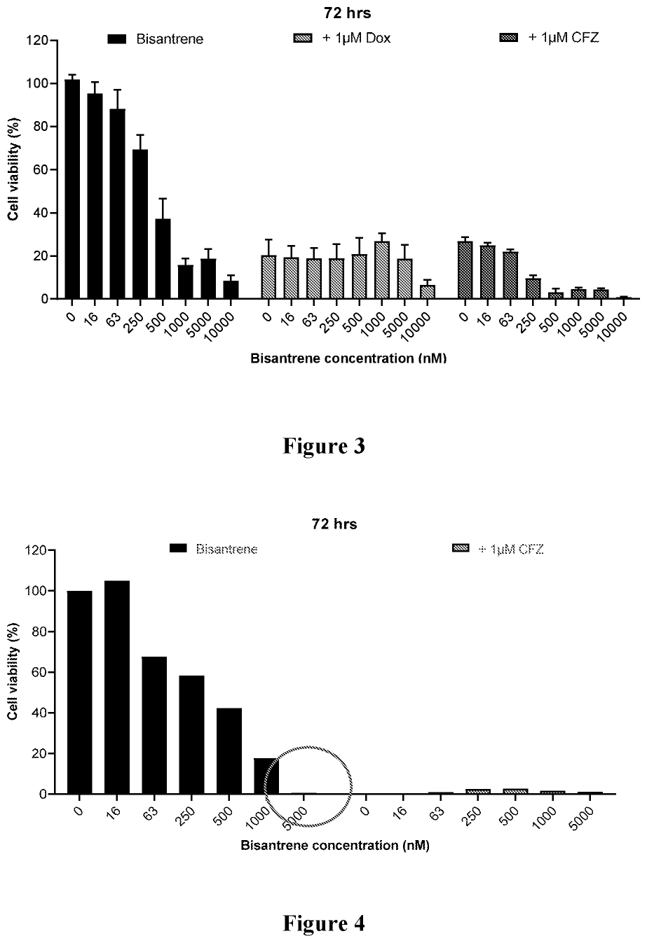

The results reported in Example 2, being protection of cardiomyocytes against toxicity of cardiotoxic agents, were surprising in view of previous reports of efficacy of bisantrene against a range of cancer cells. As shown in FIGS. 3 and 4 those reports were confirmed, illustrating results from experiments showing concentration-dependent toxicity of bisantrene to MCF7 breast cancer cells and H929 multiple myeloma cells. Furthermore, as shown in FIGS. 3 and 4 those experiments showed that bisantrene did not interfere with the toxicity of doxorubicin or carfilzomib and showed additive toxicity of bisantrene to at least MCF7 breast cancer cells (as compared to increased protection afforded to cardiomyocytes).

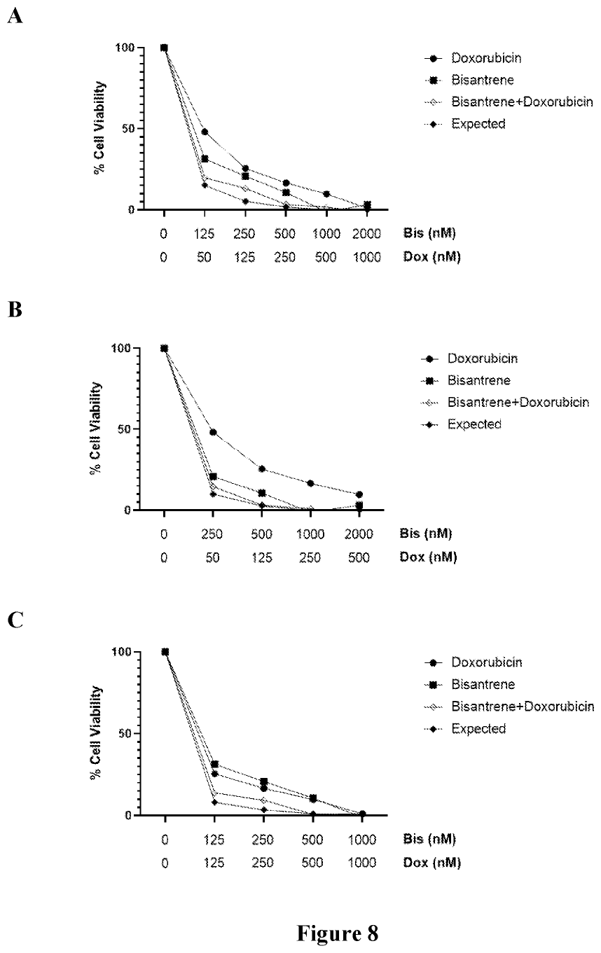

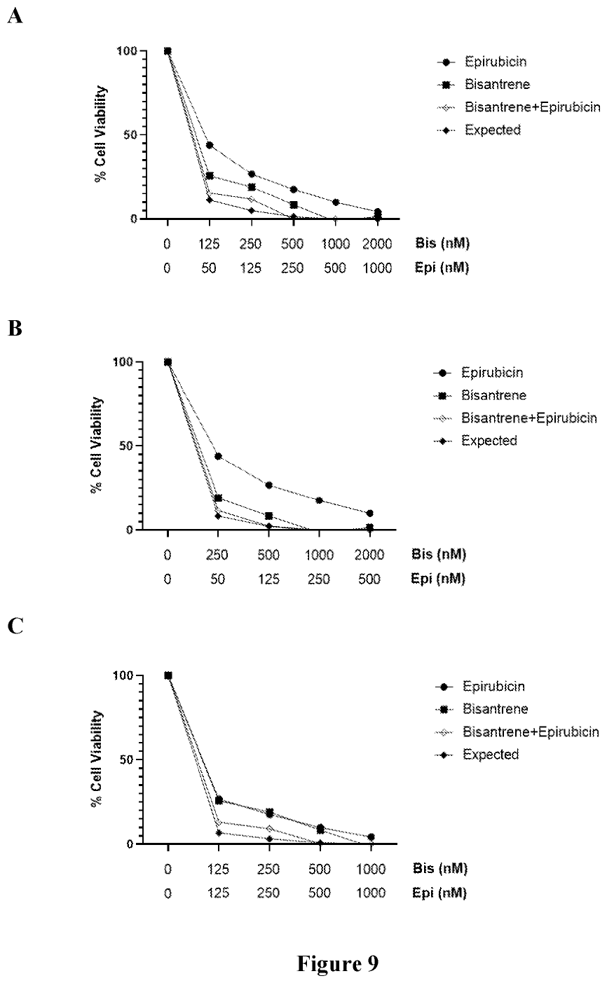

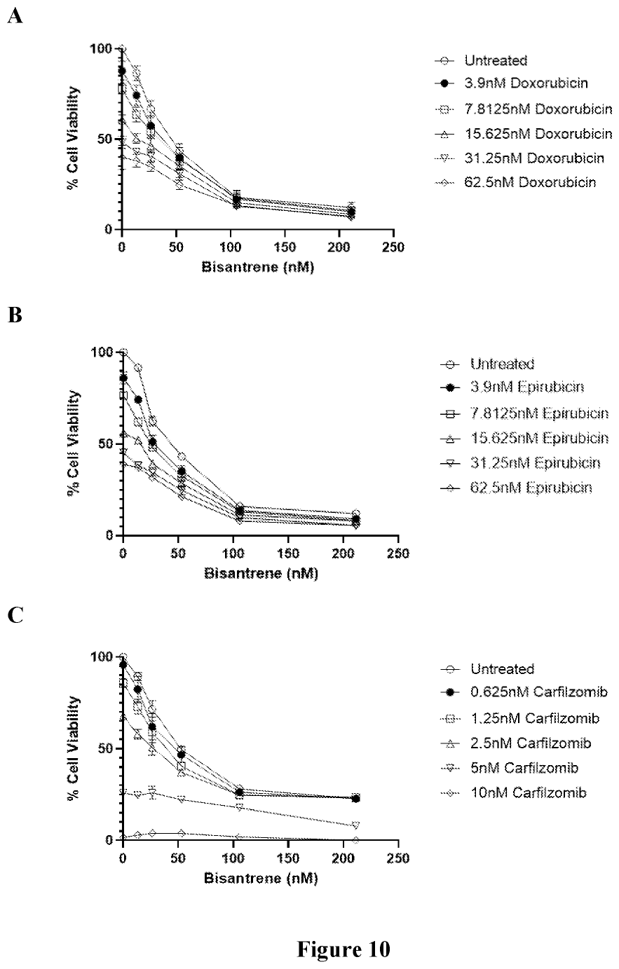

When tested against MDA-MB-231 breast cancer cells, the concentration-dependent toxic effects of bisantrene against breast cancer cells were again confirmed, and also found to be at least additive to, if not synergistic with the effects of doxorubicin, epirubicin or carfilzomib when the data was analysed via a Webb synergy analysis (see FIGS. 8 to 10), with the enhancement in combined activities appearing to be more pronounced at lower concentrations of both drugs.

### Example 4—Bisantrene Effects May not be Associated with its FTO Inhibitor Properties

Bisantrene is a compound with direct cytotoxic action as well as genomic and immunologic mechanisms of action, including as an inhibitor (IC₅₀ 142 nM) of the fat mass and obesity-associated protein (FTO), an RNA N6-methyladenosine (m6A) demethylase (Su, R., Dong, L., Li, Y., Gao, M., Han, L., Wunderlich, M., et al. (2020). Targeting FTO Suppresses Cancer Stem Cell Maintenance and Immune Evasion. Cancer Cell, 38 (1), 79-96.e11).

A number of experiments were carried out to investigate whether the cardioprotective properties of bisantrene, as well as its additive toxic effects against cancer cells (when combined with doxorubicin), were attributable to its FTO inhibitor activity.

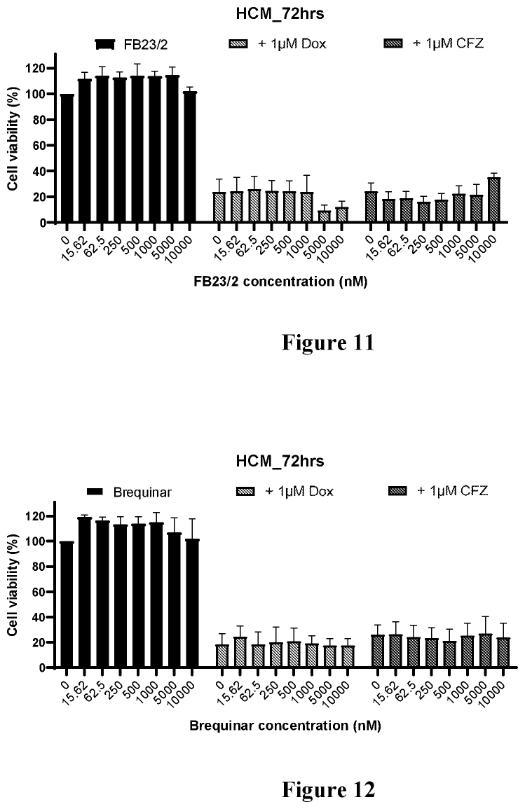

Primary HCMs were cultured in the presence of the known FTO inhibitors brequinar and FB23/2, whether alone or in combination with doxorubicin or carfilzomib. As shown in FIGS. 11 and 12, neither brequinar or FB23/2 showed appreciable toxicity towards primary HCMs cultured in their presence for up to 72 hours at FTO inhibitor concentrations of up to 10 μM. However, as also shown in FIGS. 11 and 12, unlike bisantrene, those FTO inhibitors did not mitigate the toxicity of doxorubicin or carfilzomib towards primary HCMs.

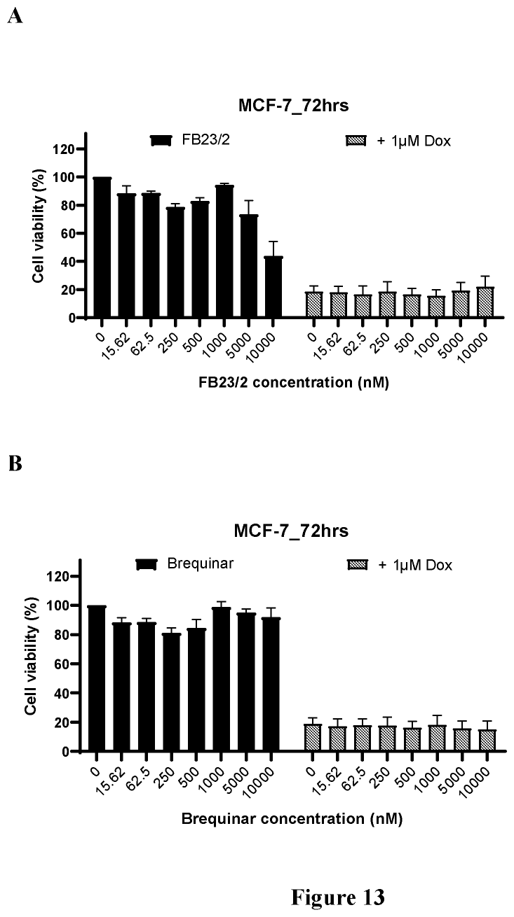

MCF7 breast cancer cells and H929 multiple myeloma cells were also cultured in the presence of the known FTO inhibitors brequinar and FB23/2, whether alone or in combination with doxorubicin, to investigate whether these FTO inhibitors could act in combination with doxorubicin to provide enhanced loss of cancer cell viability. As shown in FIG. 13, neither brequinar nor FB23/2, at concentrations of up to 10 μM, resulted in any increased loss of viability of MCF7 breast cancer cells or H929 multiple myeloma cells cultured in the presence of 1 μM doxorubicin for up to 72 hours.

These results suggest that, at least for primary human cardiac myocytes, the protective effect of bisantrene (against the cardiotoxicity of doxorubicin and carfilzomib) might not be due to its FTO inhibitor activity. These results also suggest that the enhanced toxic effects between bisantrene and doxorubicin or epirubicin (over 72 hours) against at least MCF7 or MDA-MB-231 breast cancer cells and H929 multiple myeloma cells may not be attributable to the FTO inhibitor activity of bisantrene.

### Example 5—Bisantrene May have an Optimum Dose or Dose Ratio for its Cardioprotective Effect when Co-Administered with Doxorubicin to Mice

The cardioprotective effects of bisantrene were further studied on mice. In particular, well established cardiotoxicity markers, including left ventricular fractional shortening, heart E/A ratio, left ventricular diastolic function, left ventricular dilatation, cardiac output, plasma triglyceride levels, plasma creatine kinase-MB (CK-MB), plasma LDH (lactate dehydrogenase) and plasma brain natriuretic peptide (BNP) were monitored over a period of up to 42 days following the start of treatments delivered weekly, four times (and therefore final treatment on day 21).

Treatments studied included:
vehicle alone
bisantrene (7.33 mg/kg)+vehicle
doxorubicin (5 mg/kg)+vehicle
vehicle+doxorubicin (5 mg/kg)+bisantrene (3.67 mg/kg) (“Dox+Bis 1:1”)
vehicle+doxorubicin (5 mg/kg)+bisantrene (7.33 mg/kg) (“Dox+Bis 1:2”)
vehicle+doxorubicin (4 mg/kg)+bisantrene (5.65 mg/kg) (“Dox+Bis 1:1*”)

Treatments were delivered to mice intravenously on days 0, 7, 14 and 21. Mice underwent echocardiography measurements at days 0, 21 and 42 to determine LVEF, FS, EDV, ESV, CO, E wave, A wave, E′, E/A, and E/E′, and blood samples were taken at days 0, 21 and 42 to assay for cardiac troponin I levels, BNP levels, CK, LDH levels and triglycerides.

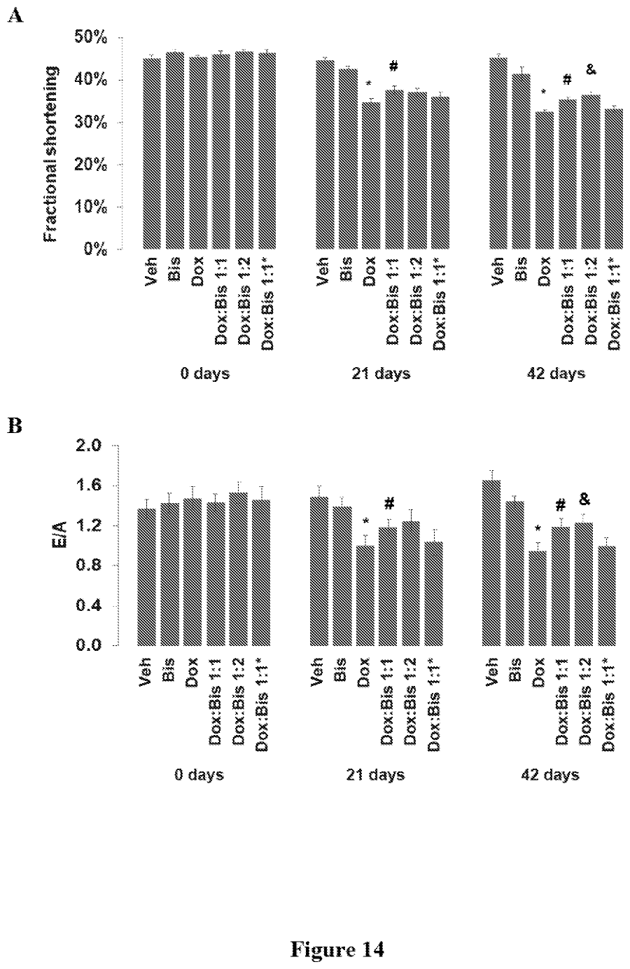

The data are presented in FIGS. 14 to 17.

As can be seen from FIG. 14, there is a suggestion (but not significant) of mild deleterious effects of bisantrene on left ventricular fractional shortening and heart E/A ratio at 7.33 mg/kg (about 22 mg/m²) per dose, whereas doxorubicin had significant cardiotoxic effects in vivo based on these parameters at 21 days after the start of treatment, with worsening by day 42. When bisantrene was co-administered with doxorubicin at 1:1 or 1:2 molar ratios, doxorubicin cardiotoxicity was significantly mitigated based on these parameters, with slightly better protection being afforded by the Dox+Bis 1:2 treatment, and less effect being observed for the Dox+Bis 80% of 1:2 treatment.

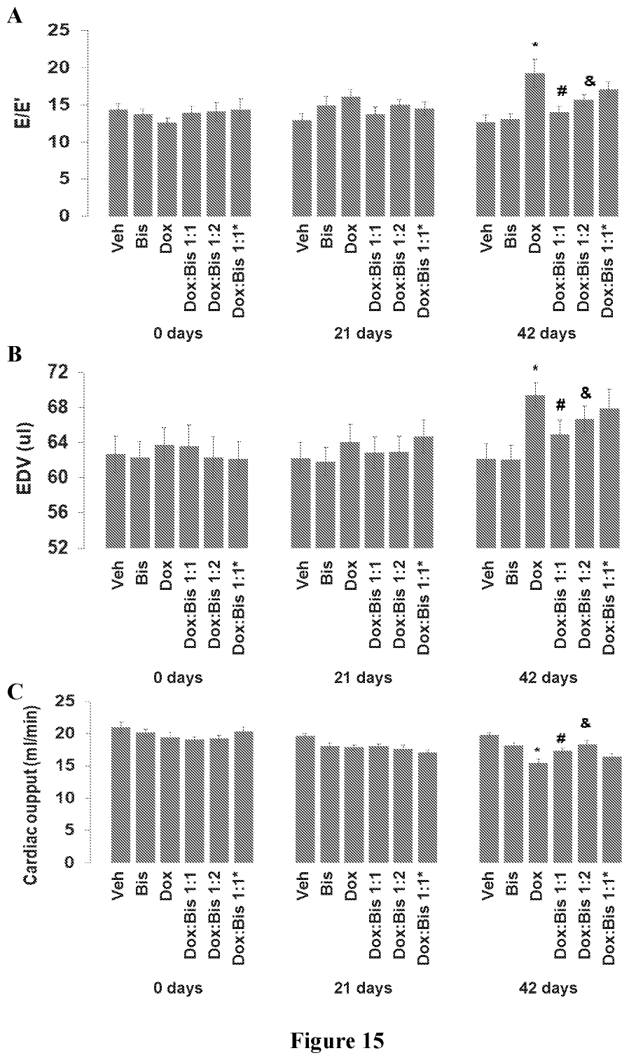

As can be seen from FIG. 15, there is a suggestion (but not significant) of mild deleterious effects of bisantrene on left ventricular diastolic function and dilatation and cardiac output at 7.33 mg/kg (about 22 mg/m²) per dose, whereas doxorubicin had significant cardiotoxic effects in vivo based on these parameters at 42 days after the start of treatment, but not significantly so by day 21. Interestingly, the Dox+Bis 1:1 treatment appeared to mitigate the cardiotoxic effects of doxorubicin, as measured by left ventricular diastolic function and dilatation as compared to both other combined treatments (which had higher bisantrene dosage rates). When looking at cardiac output, mitigation of doxorubicin cardiotoxicity was slightly greater for the Dox+Bis 1:2 treatment as compared to the Dox+Bis 1:1 treatment, with even less effect being observed for the Dox+Bis 80% of 1:2 treatment.

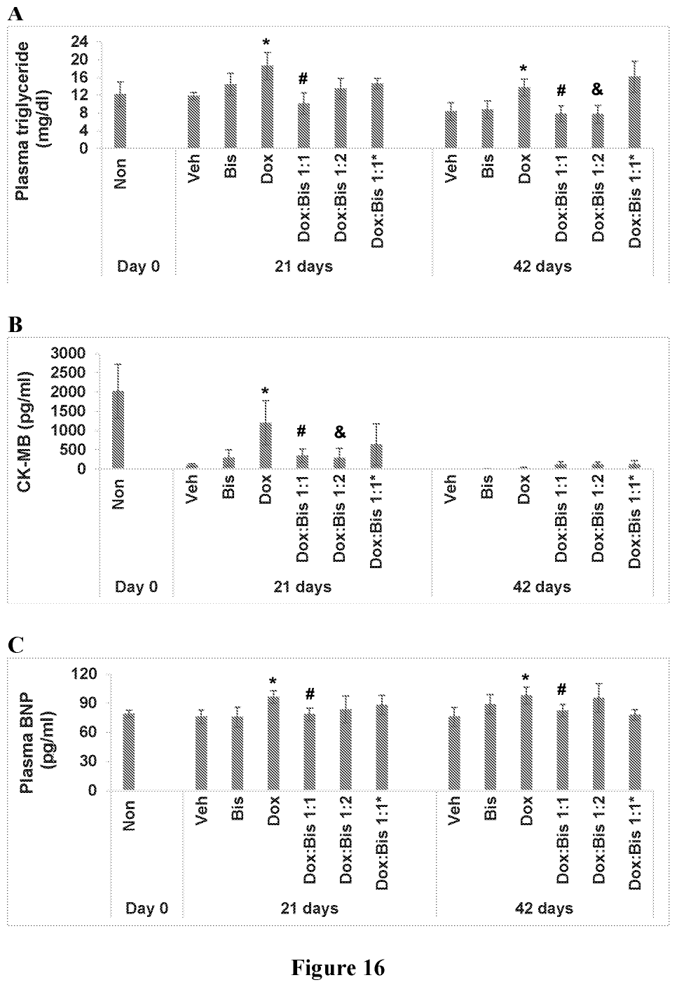

FIG. 16 shows the significant results arising from the blood tests.

FIG. 16A shows a slight (but insignificant) increase in plasma triglycerides at day 21 for the bisantrene only treatment, but not at day 42 compared to the vehicle-only control, whereas the doxorubicin treatments resulted in significantly elevated plasma triglyceride levels at day 21 as well as day 42. Co-administration of bisantrene in the Dox+Bis 1:1 treatment resulted in significant mitigation of doxorubicin-induced plasma triglyceride increases at day 21, and at day 42 for both the Dox+Bis 1:1 and Dox+Bis 1:2 treatments, and less effect being observed for the Dox+Bis 80% of 1:2 treatment.

As shown in FIG. 16B, similar effects were shown for brain natriuretic peptide levels, although results for the Dox: Bis 1:2 treatment, in terms of mitigation of any effects induced by doxorubicin, were not significant at day 42 or day 21.

FIG. 16C shows results for creatine kinase-MB (CK-MB) levels. Doxorubicin alone results in a significant increase in plasma CK-MB at day 21 while co-administration of bisantrene with doxorubicin, as both the Dox+Bis 1:1 and Dox+Bis 1:2 treatments, kept CK-MB levels down at or near the vehicle-only control levels. CK-MB levels were not significant for any treatment at day 42.

None of the co-administration treatments appeared to have any significant effect on plasma LDH levels, eosinophil, neutrophil, basophil or lymphocyte levels or viabilities, haematocrit or haemoglobin levels, mean corpuscular volume, platelet, red or white blood cell counts, or heart weight/body weight ratio.

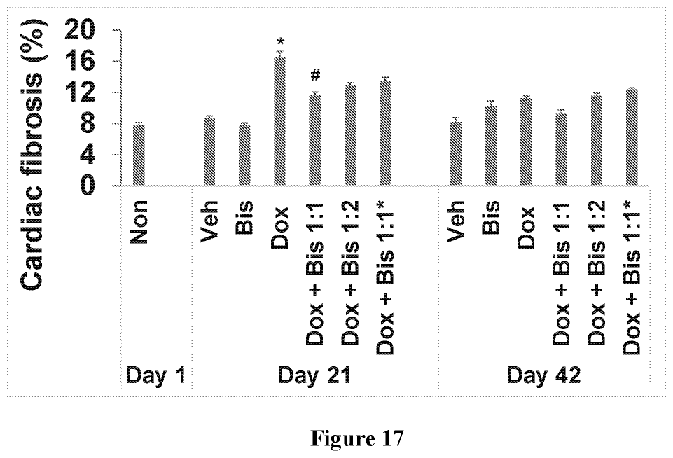

As shown in FIG. 17, bisantrene co-administered with doxorubicin at a 1:1 molar ratio significantly decreased histologic cardiac fibrosis at Day 21 relative to mice that received matched doses of doxorubicin. This result supports the functional echocardiographic and blood biomarker results, demonstrating that bisantrene was able to diminish an accepted structural marker of anthracycline-induced cardiac tissue damage.

Overall, bisantrene appeared to increase left ventricular systolic (FS) and diastolic function (E/A, E/E′), and improved cardiac function (CO, BNP) in doxorubicin-treated mice. This effect might be caused by decreasing early myocardial injury (CK-MB and cardiac fibrosis).

In this particular study, the protective effect of bisantrene appeared maximal, when considering all factors, when it was used in a 1:1 ratio with doxorubicin (or at a dosage of about 3.67 mg/m²), although the combined Dox: Bis 1:2 treatment did mitigate certain doxorubicin cardiotoxicity marker parameters more significantly than the Dox: Bis 1:1 treatment.

### CONCLUSIONS

The present studies have surprisingly shown that bisantrene is not only toxic to cancer cells, and may act in an at least additive if not synergistic manner against cancer cells when combined with doxorubicin, epirubicin or carfilzomib, but that it also mitigates toxicity of cardiotoxic therapeutic substances such as doxorubicin, daunorubicin, epirubicin, carfilzomib and bortezomib against cardiomyocytes. This latter result potentially opens the door for not only improved therapies against various cancer types, but also greater tolerance of patients to administration of cardiotoxic therapeutic agents, or longer/greater exposure of patients to such agents.

1. A method for preventing or reducing drug-induced cardiotoxicity in a subject caused by a cardiotoxic agent, the method comprising administering to the subject an effective amount of a cardioprotective agent comprising bisantrene or a derivative thereof, or a pharmaceutically acceptable salt of bisantrene or derivative thereof.

2. The method of claim 1, wherein the method comprises administering to said subject said cardioprotective agent prior to, simultaneously with, or after administration of a cardiotoxic agent to said subject.

3. The method of claim 1, wherein the method comprises administering:
a) an effective amount of a cardiotoxic chemotherapeutic agent; and
b) an effective amount of a cardioprotective agent comprising bisantrene or a derivative thereof, or a pharmaceutically acceptable salt of bisantrene or derivative thereof.

4. The method of claim 3, wherein the cancer is selected from the group consisting of breast cancer, myeloma, acute myeloid leukemia, melanoma and clear cell renal carcinoma.

5. The method of claim 2, wherein the cardiotoxic agent and the cardioprotective agent, are administered at the same time and in a single composition.

6. The method of claim 1, wherein the cardiotoxic agent is an anthracycline or pharmaceutically acceptable salt thereof.

7. The method of claim 6, wherein the anthracycline is selected from the group consisting of daunorubicin, doxorubicin, epirubicin, idarubicin, mitoxantrone, and valrubicin.

8. The method of claim 7, wherein the anthracycline is doxorubicin, daunorubicin or epirubicin.

9. The method of claim 1, wherein the cardiotoxic agent is a proteasome inhibitor, optionally carfilzomib or bortezomib.

10. The method of claim 6, wherein the dose of said cardiotoxic agent is at least 10% lower than the dose required of said cardiotoxic agent when administered without said cardioprotective agent to achieve the same targeted outcome.

11. The method of claim 10, wherein the dose of cardiotoxic agent and cardioprotective agent is in a molar ratio of from about 1:3 to about 3:1.

12. The method of claim 1, wherein the dose of cardiotoxic agent and cardioprotective agent is in a molar ratio of from about 1:1.

13. The method of claim 1, wherein the dose of cardioprotective agent is from about 5 mg/m²/week to about 100 mg/m²/week over four weeks.

14. The method of claim 1, wherein said cardioprotective agent is bisantrene or a pharmaceutically acceptable salt thereof.

15. The method of claim 14, wherein bisantrene is administered to said subject at a dosage of from about 5 mg/m²/week to about 50 mg/m²/week over four weeks, or a pharmaceutically acceptable salt of bisantrene is administered at a molar equivalent dosage rate with the same timing, and wherein said cardiotoxic agent is an anthracycline selected from the group consisting of daunorubicin, doxorubicin, epirubicin, idarubicin, mitoxantrone, and valrubicin.

16. A pharmaceutical composition comprising a cardioprotective agent comprising bisantrene or a derivative thereof, or a pharmaceutically acceptable salt of bisantrene or derivative thereof, and a cardiotoxic therapeutic agent.

17. The composition of claim 16, wherein the cardiotoxic therapeutic agent is a an anthracycline selected from the group consisting of daunorubicin, doxorubicin, epirubicin, idarubicin, mitoxantrone, and valrubicin, or a pharmaceutically acceptable salt thereof or wherein the cardiotoxic agent is a proteasome inhibitor, optionally carfilzomib or bortezomib.

18. (canceled)

19. (canceled)

20. (canceled)

21. (canceled)

22. (canceled)

23. (canceled)

24. (canceled)

25. (canceled)

26. (canceled)

27. The composition of claim 16, wherein the composition is adapted to deliver bisantrene to a subject at a dosage of from about 5 mg/m²/week to about 50 mg/m²/week over four weeks, or a pharmaceutically acceptable salt of bisantrene at a molar equivalent dosage rate with the same timing, and wherein said cardiotoxic agent is an anthracycline selected from the group consisting of daunorubicin, doxorubicin, epirubicin, idarubicin, mitoxantrone, and valrubicin.

28. A kit for preventing or reducing drug-induced cardiotoxicity in a subject caused by a cardiotoxic therapeutic agent, said kit comprising a cardioprotective agent comprising bisantrene or a derivative thereof, or a pharmaceutically acceptable salt of bisantrene or derivative thereof, and said cardiotoxic therapeutic agent.

29. The kit of claim 28, wherein the kit is for treating a subject with cancer.
